(19) United States
(12) Patent Application Publication
(10) Pub. No.: US 2023/0285543 A1
(43) Pub. Date: Sep. 14, 2023

(54) PEPTIDE EPITOPE VACCINES FOR COVID-19 AND METHOD OF DESIGNING, MAKING AND USING THE SAME

(71) Applicant: UNIVERSITY OF SOUTHERN CALIFORNIA, Los Angeles, CA (US)

(72) Inventors: Paul BOGDAN, Los Angeles, CA (US); Shahin NAZARIAN, Los Angeles, CA (US); Zikun YANG, Los Angeles, CA (US)

(73) Assignee: UNIVERSITY OF SOUTHERN CALIFORNIA, Los Angeles, CA (US)

(21) Appl. No.: 18/016,230

(22) PCT Filed: Jul. 14, 2021
(86) PCT No.: PCT/IB2021/056355

§ 371 Date: Jan. 13, 2023

Related U.S. Application Data
(60) Provisional application No. 63/052,321, filed on Jul. 15, 2020.

(51) Int. Cl. A61K 39/215; A61P 31/14; G16B 15/20; G16B 15/30

(52) U.S. Cl. CPC A61K 39/215; A61P 31/14; G16B 15/20; G16B 15/30

(57) ABSTRACT

Computer systems and computer implemented methods are presented for designing and making vaccines to pathogens, particular viral pathogens. Vaccine compositions for COVID-19 are also disclosed, as well as method of using the same.

### CROSS-REFERENCE TO RELATED APPLICATION

This patent application is the National Phase of International Application No. PCT/IB2021/056355, filed Jul. 14, 2021, which claims the benefit of priority to U.S. Provisional Patent Application No. 63/052,321, filed Jul. 15, 2020. The entire contents of the foregoing applications are incorporated herein by reference, including all text, tables and drawings.

### GOVERNMENT SUPPORT

This invention was made with government support under institute contract/grant number CPS/CNS-1453860 awarded by National Science Foundation (NSF) and institute contract/grant number N66001-17-1-4044 awarded by U.S. Army Defense Advanced Research Projects Agency (DARPA). The government has certain rights in the invention.

### FIELD OF THE INVENTION

Provided herein, in certain aspects, are systems and methods of designing and making a vaccine. Provided herein, in certain aspects, are compositions and methods for inhibiting or preventing viral infections.

### BACKGROUND

The rampant spread of COVID-19, an infectious disease caused by SARS-CoV-2, all over the world has led to over 4.5 million cases and more than 300,000 deaths, and devastated the social, financial, and political entities around the world. Without an existing effective medical therapy, vaccines are urgently needed to avoid the spread of this and other pathogenic diseases.

### SUMMARY

Presented herein, in part, are in-silico (e.g., computer based and/or otherwise computational) deep learning approaches for developing epitope-based peptide vaccines. By comprehensively investigating existing public databases, the proposed deep learning framework directly predicts epitope vaccine sequences from SARS-CoV-2 spike proteins using a deep neural network.

By employing in-silico methods to predict linear B-cell epitopes, human-leukocyte-antigen (HLA) restricted T-cell epitopes and protective antigenicity, 130 candidate epitopes were initially identified and further analyzed to provide the best vaccine candidates. This analysis predicted 20 B-cell epitopes and 187 T-cell epitopes and identified 14 optimal vaccine candidates. The toxicity, physicochemical properties and allergenicity of the vaccine candidates were also evaluated in order to determine their safety risks, side effects and practical applications. The proposed artificial intelligence vaccine discovery framework accelerates the vaccine design process. RNA mutation of the CoV are also used to design two additional vaccine candidates to address viral mutations.

Also, presented herein are compositions comprising one or more peptide epitopes from SARS-CoV-2, which composition may comprise an adjuvant, and method of using the same.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 shows a conventional in-silico vaccine design process. Overlapping proteins are all the continuous protein sequence with the same length in the whole protein sequence. Further evaluation includes physicochemical analysis, side effect evaluation, clinical trials. There are many intermittent and inefficient middle steps during this vaccine design process.

FIG. 2 show an embodiment of an in-silico Vaccine Design with the present systems and methods. By combining many intermittent middle steps into one deep neural network (DNN), the vaccine evaluation is allowed to initiate from a much smaller amount of candidates.

FIG. 3 shows Receiver operating characteristic (ROC) curves.

FIG. 4 is a diagram that illustrates an exemplary computing system in accordance with embodiments of the present systems and methods.

FIG. 5 is another schematic diagram of an in-silico vaccine design process showing a traditional design process (A) compared to a design process (B) using the present systems and methods.

FIG. 6 illustrates surface accessibility of SARS-CoV-2.

FIG. 7 is a schematic presentation of a present multi-epitope vaccine.

FIG. 8 is a graphical representation of secondary structure features.

FIG. 9 illustrates solvent accessibility and disorder regions prediction results.

FIG. 10 illustrates a vaccine three-dimensional structure.

FIG. 11 illustrates a refined vaccine three dimensional structure.

FIG. 12 illustrates vaccine three dimensional structure validation.

FIG. 13 illustrates a model of six predicted conformational B-cell epitopes in a refined final vaccine structure.

FIG. 14 illustrates vaccine in-silico cloning into a pET28a(+) vector.

FIG. 15 illustrates a docked complex of a present vaccine model and the TLR4 immune receptor.

FIG. 16 illustrates a molecular dynamics simulation of a vaccine TLR4 docked complex.

### DETAILED DESCRIPTION

To mitigate the problems described herein, the inventors had to both invent solutions and, in some cases just as importantly, recognize problems overlooked (or not yet foreseen) by others in the field of vaccine design. Indeed, the inventors wish to emphasize the difficulty of recognizing those problems that are nascent and will become much more apparent in the future should trends in industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described below.

Presented herein, in some embodiments, are in-silico (e.g., computer based and/or otherwise computational) methods of vaccine design and vaccines that can be used to prevent pathogen infections, particularly viral infections.

SARS-CoV-2

Coronavirus disease 2019 (COVID-19) is an infectious disease caused by severe acute respiratory syndrome coronavirus 2 (SARS-CoV-2)^{1, 2}. First detected in December 2019 in Wuhan, the virus has spread globally, resulting in over 4.5 million infected cases, more than 300,000 deaths³, and unprecedented financial, social and political impacts all over the world⁴. Efficacious vaccines are therefore desperately needed. Main clinical features of the COVID-19 are fever, cough and myalgia or fatigue⁶; the virus has caused clusters of severe respiratory illness similar to severe acute respiratory syndrome coronavirus and is associated with ICU admission and high mortality⁷.

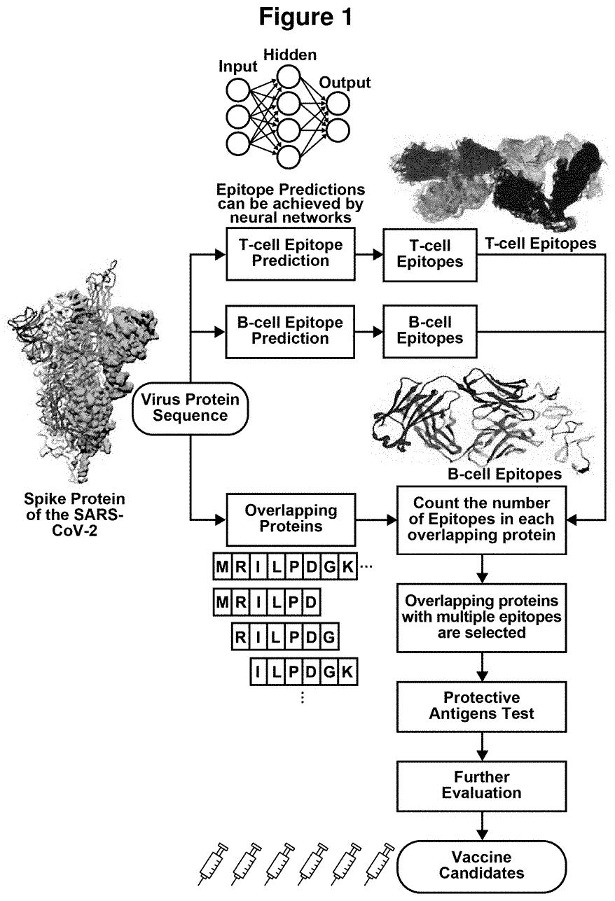

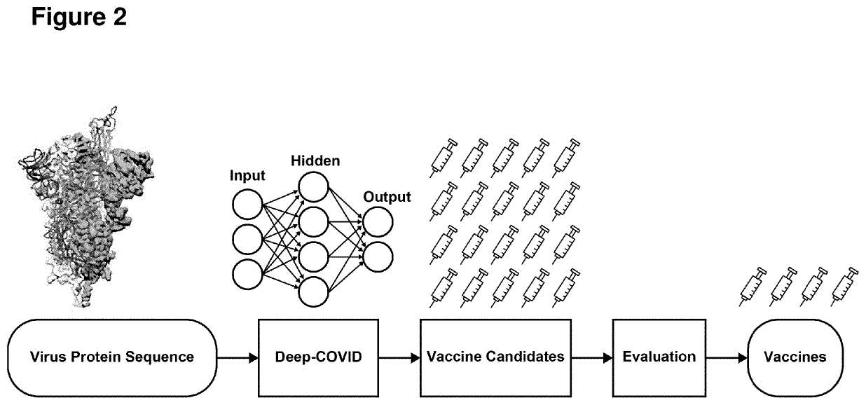

Currently, without a single specific antiviral therapy for CoV, the control methods of the COVID-19 are early diagnosis, reporting, isolation, supportive treatments, and timely publishing epidemic information with only limited impact on the coronavirus^{8, 9}. Researchers have proposed several approaches to develop vaccines for the CoV10. Traditional process of vaccine design is based on growing pathogens, which has a very time-consuming process of isolating, inactivating and injecting the virus that cause the disease^{1, 12}. Such process usually takes more than a year to result in efficacious vaccines and hence contributes very little to avoid the current spread of the disease^{13, 14}. Recently, researchers have analyzed the virus protein sequences by in-silico methods to reduce the potential vaccine candidates without the need to grow pathogens to accelerate the vaccine design process¹⁵. With the completion of the genome sequencing of the SARS-CoV-28, many peptide vaccines are selected out based on the epitopes predicted in certain areas of the virus protein sequences, by approaches such as machine learning (ML), sequence motif, motif matrix, etc.¹⁶. Ideal peptide vaccines are small fragments of the viral protein sequence containing both the B-cell epitopes and the T-cell epitopes and the whole vaccine peptide sequence must be a protective antigen to have the ability to trigger human protective reactions^{17, 18}. Consequently, an in-silico vaccine design process usually has three main steps which are B-cell epitopes prediction, T-cell epitopes prediction and the protective antigens prediction¹⁹. FIG. 1 illustrates a typica process, and FIG. 2 provides a schematic diagram example of the newly proposed in-silico vaccine design processes. As shown in FIG. 2, by combining many intermittent middle steps into one deep neural network (DNN), the vaccine evaluation is allowed to initiate from a much smaller amount of candidates. The three main prediction steps can be performed by supervised learning utilizing neural network based classifiers²⁰. Towards this end, the in-silico vaccine design was conducted on the spike protein area of the CoV²¹. By using a deep neural network (DNN) with subtly designed structures trained by a combination of the protein sequences from well-known online prediction tools, higher efficiency, better accuracy, and a list of potential vaccine candidates are provided.

Subjects

The term “subject” refers to a mammal. Any suitable mammal can be treated by a method or composition described herein. Non-limiting examples of mammals include a human, non-human primate (e.g., ape, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a subject is a non-human primate or a human In some embodiments a subject is a human. A subject can be any age or at any stage of development (e.g., an adult, teen, child, infant, or a mammal in utero). A subject can be male or female.

In some embodiments, a subject has, is suspected of having, or is at risk of having an infection from a pathogen. In certain embodiments, a subject at risk of having an infection from a pathogen is a subject at risk of acquiring a viral infection. In certain embodiments, a subject at risk of acquiring a viral infection is a subject at risk of acquiring SARS-CoV-2. A subject at risk of acquiring a viral infection is a subject who may come into contact with another who can potentially transmit the viral infection to the subject.

Compositions

Disclosed herein are compositions that can be used to stimulate an immune response in a subject. In certain embodiments, a composition comprises 1 or more, 2 or more, 3 or more, 4 ore more or 5 or more peptides derived from a pathogen (e.g., a viral pathogen). In some embodiments, a compositions comprises one or more peptides comprising 5 or more contiguous amino acids of a peptide selected from Tables 3-11. In some embodiments, a compositions comprises one or more peptides comprising 6 or more, 7 or more, 8 or more, 10 or more, 11 or more, 15 or more or 20 or more contiguous amino acids of a peptide selected from Tables 3-11.

In some embodiments, a compositions comprises one or more peptides, each peptide having at least 80%, at least 85%, at least 90%, at least 95%, at least 96%, at least 97%, at least 98%, or at least 99% identity to peptide selected from Tables 3-11. In some embodiments, a compositions comprises one or more peptides, each peptide having at least 95% identity to peptide selected from Tables 3-11.

The term “percent identical” or “percent identity” refers to sequence identity between two amino acid sequences. Identity can be determined by comparing a position in each sequence which may be aligned for purposes of comparison. When an equivalent position in the compared sequences is occupied by the same amino acid, then the molecules are identical at that position. When the equivalent site is occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

In some embodiments, a composition comprises one or more peptides selected from Tables 3-11. In some embodiments, a composition comprises one or more peptides selected from Tables 3-11 and a suitable adjuvant.

In some embodiments, a composition disclosed herein induces an immune response in a subject. An immune response may include a T-cell response or a B-cell response. In certain embodiments, a composition induces an immune response to one more viral proteins of SARS-CoV-2. In certain embodiments, an immune response comprises production of antibodies that specifically bind to one more viral proteins of SARS-CoV-2.

In some embodiments a composition is a vaccine composition. In some embodiments, a vaccine composition is a pharmaceutical composition. In some embodiments, a composition or pharmaceutical composition comprises an immunogenic amount of one or more peptide epitopes disclosed herein. In some embodiments, a composition or pharmaceutical composition comprises one or more peptide epitopes in an amount in a range of 1 μg to 100 mg, or 10 μg to 10 mg. In some embodiments provided herein is a pharmaceutical composition comprising a one or more peptide epitopes disclosed herein for use in conducting a method described herein. In some embodiments, a pharmaceutical composition comprises a composition disclosed herein and a pharmaceutically acceptable excipient, diluent, additive or carrier.

A pharmaceutical composition can be formulated for a suitable route of administration. In some embodiments a pharmaceutical composition is formulated for oral, subcutaneous (s.c.), intradermal, intramuscular, intraperitoneal and/or intravenous (i.v.) administration. In certain embodiments, a pharmaceutical composition contains formulation materials for modifying, maintaining, or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates (e.g., phosphate buffered saline) or suitable organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); solvents (such as glycerin, propylene glycol or polyethylene glycol); diluents; excipients and/or pharmaceutical adjuvants. In particular, a pharmaceutical composition can comprise any suitable carrier, formulation, or ingredient, the like or combinations thereof as listed in “Remington: The Science And Practice Of Pharmacy” Mack Publishing Co., Easton, Pa., 19th Edition, (1995)(hereafter, Remington '95), or “Remington: The Science And Practice Of Pharmacy”, Pharmaceutical Press, Easton, Pa., 22^{nd} Edition, (2013)(hereafter, Remington 2013), the contents of which are incorporated herein by reference in their entirety.

In certain embodiments, a pharmaceutical composition comprises a suitable excipient, non-limiting examples of which include anti-adherents (e.g., magnesium stearate), a binder, fillers, monosaccharides, disaccharides, other carbohydrates (e.g., glucose, mannose or dextrin), sugar alcohols (e.g., mannitol or sorbitol), coatings (e.g., cellulose, hydroxypropyl methylcellulose (HPMC), microcrystalline cellulose, synthetic polymers, shellac, gelatin, corn protein zein, enterics or other polysaccharides), starch (e.g., potato, maize or wheat starch), silica, colors, disintegrants, flavors, lubricants, preservatives, sorbents, sweeteners, vehicles, suspending agents, surfactants and/or wetting agents (such as pluronics, PEG, sorbitan esters, polysorbates such as polysorbate 20, polysorbate 80, triton, tromethamine, lecithin, cholesterol, tyloxapal), stability enhancing agents (such as sucrose or sorbitol), and tonicity enhancing agents (such as alkali metal halides, sodium or potassium chloride, mannitol, sorbitol), and/or any excipient disclosed in Remington '95 or Remington 2013. The term “binder” as used herein refers to a composition or ingredient that helps keeps a pharmaceutical mixture combined. Suitable binders for making pharmaceutical formulations and are often used in the preparation of pharmaceutical tablets, capsules and granules are known to those skilled in the art.

In some embodiments a pharmaceutical composition comprises a suitable pharmaceutically acceptable additive and/or carrier. Non-limiting examples of suitable additives include a suitable pH adjuster, a soothing agent, a buffer, a sulfur-containing reducing agent, an antioxidant and the like. Non-limiting examples of a sulfur-containing reducing agent include those having a sulfhydryl group (e.g., a thiol) such as N-acetylcysteine, N-acetylhomocysteine, thioctic acid, thiodiglycol, thioethanolamine, thioglycerol, thiosorbitol, thioglycolic acid and a salt thereof, sodium thiosulfate, glutathione, and a C1-C7 thioalkanoic acid. Non-limiting examples of an antioxidant include erythorbic acid, dibutylhydroxytoluene, butylhydroxyanisole, alpha-tocopherol, tocopherol acetate, L-ascorbic acid and a salt thereof, L-ascorbyl palmitate, L-ascorbyl stearate, sodium bisulfite, sodium sulfite, triamyl gallate and propyl gallate, as well as chelating agents such as disodium ethylenediaminetetraacetate (EDTA), sodium pyrophosphate and sodium metaphosphate. Furthermore, diluents, additives and excipients may comprise other commonly used ingredients, for example, inorganic salts such as sodium chloride, potassium chloride, calcium chloride, sodium phosphate, potassium phosphate and sodium bicarbonate, as well as organic salts such as sodium citrate, potassium citrate and sodium acetate.

The pharmaceutical compositions used herein can be stable over an extended period of time, for example on the order of months or years. In some embodiments a pharmaceutical composition comprises one or more suitable preservatives. Non-limiting examples of preservatives include benzalkonium chloride, benzoic acid, salicylic acid, thimerosal, phenethyl alcohol, methylparaben, propylparaben, chlorhexidine, sorbic acid, hydrogen peroxide, the like and/or combinations thereof. A preservative can comprise a quaternary ammonium composition, such as benzalkonium chloride, benzoxonium chloride, benzethonium chloride, cetrimide, sepazonium chloride, cetylpyridinium chloride, or domiphen bromide (BRADOSOL®). A preservative can comprise an alkyl-mercury salt of thiosalicylic acid, such as thimerosal, phenylmercuric nitrate, phenylmercuric acetate or phenylmercuric borate. A preservative can comprise a paraben, such as methylparaben or propylparaben. A preservative can comprise an alcohol, such as chlorobutanol, benzyl alcohol or phenyl ethyl alcohol. A preservative can comprise a biguanide derivative, such as chlorohexidine or polyhexamethylene biguanide. A preservative can comprise sodium perborate, imidazolidinyl urea, and/or sorbic acid. A preservative can comprise stabilized oxychloro complexes, such as known and commercially available under the trade name PURITE®. A preservative can comprise polyglycol-polyamine condensation resins, such as known and commercially available under the trade name POLYQUART® from Henkel KGaA. A preservative can comprise stabilized hydrogen peroxide. A preservative can be benzalkonium chloride. In some embodiments a pharmaceutical composition is free of preservatives.

In some embodiments a composition disclosed herein is substantially free of contaminants (e.g., blood cells, platelets, polypeptides, minerals, blood-borne compositions or chemicals, virus, bacteria, other pathogens, toxin, and the like). In some embodiments a composition or pharmaceutical composition disclosed herein is substantially free of serum and serum contaminants (e.g., serum proteins, serum lipids, serum carbohydrates, serum antigens and the like). In some embodiments a composition or pharmaceutical composition disclosed herein is substantially free of a pathogen (e.g., an whole, intact, live or attenuated virus, parasite or bacteria). In some embodiments a composition or pharmaceutical composition disclosed herein is sterile.

The pharmaceutical compositions described herein may be configured for administration to a subject in any suitable form and/or amount according to the use in which they are employed. For example, a pharmaceutical composition configured for parenteral administration (e.g., by injection or infusion), may take the form of a suspension, solution or emulsion in an oily or aqueous vehicle and it may contain formulation agents, excipients, additives and/or diluents such as aqueous or non-aqueous solvents, co-solvents, suspending solutions, preservatives, stabilizing agents and or dispersing agents. In some embodiments a pharmaceutical composition suitable for parenteral administration may contain one or more excipients. In some embodiments a pharmaceutical composition is lyophilized to a dry powder form. In some embodiments a pharmaceutical composition is lyophilized to a dry powder form, which is suitable for reconstitution with a suitable solvent (e.g., water, oil, saline, an isotonic buffer solution (e.g., PBS), DMSO, combinations thereof and the like). In certain embodiments, reconstituted forms of a lyophilized pharmaceutical composition are suitable for parenteral administration (e.g., intramuscular or subcutaneous administration) to a mammal.

In certain embodiments, an optimal pharmaceutical composition is determined by one skilled in the art depending upon, for example, on the intended route of administration, delivery format and desired dosage (see e.g., Remington '95 or Remington 2013, supra). A pharmaceutical composition can be manufactured by any suitable manner, including, e.g., by means of conventional mixing, dissolving, granulating, dragee-making, levigating, emulsifying, encapsulating, entrapping or tableting processes (e.g., see methods described in Remington '95 or Remington 2013).

In some embodiments, a composition comprises one or more suitable adjuvants, non-limiting examples of which include aluminum compounds (e.g., alum, Alhydrogel), oils, block polymers, immune stimulating complexes, vitamins and minerals (e.g., vitamin E, vitamin A, selenium, and vitamin B12), Quil A (saponins), bacterial and fungal cell wall components (e.g., lipopolysaccharides, lipoproteins, and glycoproteins), hormones, cytokines, co-stimulatory factors, the like or combinations thereof.

Route of Administration

Any suitable method of administering a composition, pharmaceutical composition or composition disclosed herein to a subject can be used. Any suitable formulation and/or route of administration can be used for administration of a composition disclosed herein or composition disclosed herein (e.g., see Fingl et al. 1975, in “The Pharmacological Basis of Therapeutics”, which is incorporated herein by reference in its entirety). A suitable formulation and/or route of administration can be chosen by a medical professional (e.g., a physician) in view of, for example, a subject's risk, age, and/or condition. Non-limiting examples of routes of administration include topical or local (e.g., transdermally or cutaneously, (e.g., on the skin or epidermis), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranial, intra-articular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments a composition disclosed herein or pharmaceutical composition described herein is administered to the lungs, bronchial passages, trachea, esophagus, sinuses, or nasal passages using a suitable method, non-limiting examples of which include intranasal administration, intratracheal instillation, and oral inhalative administration (e.g., by use of an inhaler, e.g., single/-multiple dose dry powder inhalers, nebulizers, and the like).

In some embodiments a composition disclosed herein or a pharmaceutical composition disclosed herein is provided to a subject. For example, a composition that is provided to a subject is sometimes provided to a subject for self-administration or for administration to a subject by another (e.g., a non-medical professional). As another example, a composition can be provided as an instruction written by a medical practitioner that authorizes a patient to be provided a composition or treatment described herein (e.g., a prescription).

Dose and Immunogenic Amount

In some embodiments, an amount of a composition disclosed herein (e.g., in a pharmaceutical composition) is an immunogenic amount. In certain embodiments, a pharmaceutical composition comprises an immunogenic amount of a composition disclosed herein. In some embodiments, an immunogenic amount of a composition disclosed herein is administered to a subject. In some embodiments, an immunogenic amount of a composition disclosed herein is an amount needed to induce an immune response. In some embodiments, an immunogenic amount of a composition disclosed herein is an amount needed to obtain an effective therapeutic outcome (e.g., complete or partial immunity to a virus). In certain embodiments, an immunogenic amount of a composition disclosed herein is an amount sufficient to inhibit or prevent SARS-CoV-2 infection. Determination of an immunogenic amount is well within the capability of those skilled in the art, especially in light of the detailed disclosure provided herein.

In certain embodiments, an immunogenic amount is an amount high enough to provide an immune response (e.g., a protective immune response) and an amount low enough to minimize unwanted adverse reactions. Accordingly, in certain embodiments, an immunogenic amount of a composition disclosed herein may vary from subject to subject, often depending on age, weight, general health condition of a subject and severity of a condition being treated. Thus, in some embodiments, an immunogenic amount is determined empirically. Accordingly, an immunogenic amount of a composition that is administered to a subject can be determined by one of ordinary skill in the art based on amounts found effective in animal or clinical studies, a physician's experience, and suggested dose ranges or dosing guidelines, for example.

In certain embodiments, an immunogenic amount of a composition disclosed herein is administered at a suitable dose (e.g., at a suitable volume, frequency and/or concentration, which often depends on a subject's weight, age and/or condition) intended to obtain an acceptable therapeutic outcome. In certain embodiments, an immunogenic amount of a composition comprises one or more doses selected from at least 0.001 mg/kg (e.g., mg of a composition per kg body weight of a subject), at least 0.01 mg/kg, at least 0.1 mg/kg, at least 1 mg/kg, at least 10 mg/kg or at least 100 mg/kg. In certain embodiments, an immunogenic amount of a composition is selected from one or more doses of about 0.001 mg/kg (e.g., mg of a composition per kg body weight of a subject) to about 100 mg/kg, 0.001 mg/kg to 50 mg/kg, 0.001 mg/kg to 10 mg/kg, 0.01 mg/kg to 10 mg/kg, 0.01 mg/kg to 5 mg/kg, intervening amounts and combinations thereof. In some embodiments an immunogenic amount of a composition disclosed herein is between about 0.001 mg/kg and about 50 mg/kg.

In some embodiments administering an immunogenic amount of a composition disclosed herein, or a pharmaceutical composition disclosed herein, comprises administering a suitable dose at a frequency or interval as needed to obtain an effective therapeutic outcome. In some embodiments administering an immunogenic amount of a composition or a pharmaceutical composition disclosed herein comprises administering a suitable dose once a day, twice a week, weekly, at combinations thereof, and/or at regular or irregular intervals thereof, and/or simply at a frequency or interval as needed or recommended by a medical professional.

In some embodiments, a method comprises administration of a composition disclosed herein to a subject. In certain embodiments, a method comprises inducing an immune response in a subject to one or more viral proteins, or portions thereof. In certain embodiments, a method comprises inducing an immune response in a subject to one or more proteins of SARS-CoV-2. In certain embodiments, a method comprises inducing an immune response to SARS-CoV-2.

In certain embodiments, a method comprises administering a composition disclosed herein to a subject, the composition optionally comprising a adjuvant, wherein the method reduces, inhibits, mitigates or prevents infection of the subject with SARS-CoV-2. In certain embodiments, a method comprises administering a composition disclosed herein to a subject, the composition optionally comprising a adjuvant, wherein the method reduces, inhibits, mitigates or prevents one or more symptoms of a SARS-CoV-2 infection. In certain embodiments, a method comprises administering a composition disclosed herein to a subject, the composition optionally comprising a adjuvant, wherein the method reduces, inhibits, mitigates or prevents the severity of one or more symptoms of a SARS-CoV-2 infection.

Non-limiting examples of a symptom of a SARS-CoV-2 infection include fever, chills, cough, shortness of breath, difficulty breathing, fatigue, muscle or body aches, headache, new loss of taste or smell, sore throat, congestion, runny nose, nausea, vomiting, and diarrhea.

Kits

In some embodiments, provided herein is a kit comprising a composition disclosed herein or a pharmaceutical composition comprising a composition disclosed herein. In some embodiments, a kit comprises one or more doses of a pharmaceutical composition comprising a composition disclosed herein. In some embodiments, a kit comprises one or more packs and/or one or more dispensing devices, which can contain one or more doses of a composition disclosed herein, or pharmaceutical composition thereof, as described herein. Non-limiting examples of a pack include a metal, glass, or plastic container, syringe or blister pack that comprises a composition disclosed herein or a composition described herein. In certain embodiments, a kit comprises a dispensing device such as a syringe or inhaler, that may or may not comprise a composition disclosed herein or a composition described herein. A pack and/or dispenser device can be accompanied by instructions for administration. The pack or dispenser can also be accompanied with a notice associated with the container in a form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert.

In some embodiments a kit or pack comprises an amount of a composition disclosed herein sufficient to 1-10 administrations of a compositions herein.

In some embodiments, a kit comprises a computer readable medium, optical disk such as CD- or DVD-ROM/RAM, DVD, MP3, magnetic tape, or an electrical storage media such as RAM and ROM or hybrids of these such as magnetic/optical storage media, FLASH media or memory-type cards.

A kit optionally includes a product label and/or one or more packaging inserts, that provide a description of the components or instructions for use in vitro, in vivo, or ex vivo, of the components therein. Exemplary instructions may include instructions for a treatment protocol or therapeutic regimen. In certain embodiments, a kit comprises packaging material, which refers to a physical structure housing components of the kit. The packaging material can maintain the components sterilely and can be made of material commonly used for such purposes (e.g., paper, corrugated fiber, glass, plastic, foil, ampules, vials, tubes, etc.). Product labels or inserts include “printed matter,” e.g., paper or cardboard, or separate or affixed to a component, a kit or packing material (e.g., a box), or attached to an ampule, tube or vial containing a kit component. Product labels or inserts can include identifying information of one or more components therein, dose amounts, clinical pharmacology of the active ingredient(s) including mechanism of action, pharmacokinetics (PK) and pharmacodynamics (PD). Product labels or inserts can include information identifying manufacturer information, lot numbers, manufacturer location, date, information on an indicated condition, disorder, disease or symptom for which a kit component may be used. Product labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. A kit can additionally include labels or instructions for practicing any of the methods described herein. Product labels or inserts can include information on potential adverse side effects and/or warnings.

As described above, traditional in-silico vaccine design approaches are inefficient, and are not sufficiently fast to keep pace with the emergence of various pandemics. Currently, there are some online tools for in-silico vaccine design. They are however designed to reach only one prediction out of the several needed for a reliable vaccine design process. For example, BepiPred is a very popular B-cell epitope prediction tool and many researchers use this tool to predict the B-cell epitopes. However, BepiPred can only be used to address one step of B-cell epitope prediction, and when it comes to T-cell epitope prediction, a different tool such as NetMHCpan is needed. No current tool can directly predict vaccine candidates from virus proteins. There are at least two drawbacks to this. Firstly, for example, there are too many middle steps, which create a large overhead for moving intermediate results from one tool to another. Secondly, for example, for parallel steps like T-cell and B-cell epitope predictions, they are done on the whole sequence, resulting in unnecessary prediction computations, whereas only the common or the near parts of the sequence should be analyzed for those two predictions steps.

An efficient vaccine tool (framework) is presented herein and based on a single DNN that can quickly and efficiently deduce a relatively small number of vaccine candidates directly from viral protein sequences, thereby allowing refined in-silico methods of vaccine design to initiate from a much smaller amount of data with much higher efficiency.

Data Collection and Dataset Design

Reliable data is essential for the performance of supervised learning²⁴, thus it plays a crucial role in the outcome of the vaccine design process. Several (5000) known B-cell epitopes (B) and 2000 known T-cell epitopes containing both MHC (major histocompatibility complex)-1 and MHC-2 binders²⁵ (T) from the NCBI database were collected, and combined with the same number of proteins which are not T-cell or B-cell epitopes, forming a dataset of epitopes and non-epitopes. Several (100) known latest viral protective antigens were selected from the NCBI database, and the same number of proteins without protective functions were randomly selected, combining with the 400 antigens in previous work, forming a dataset with 600 antigens.

The present systems, methods, and/or vaccines are built based on supervised learning on a specifically designed dataset. To directly predict vaccine candidates, the protein sequences in the positive dataset contain at least one T-cell epitope and one B-cell epitope and are protective antigens. A Cartesian Product is the set that contains all ordered pairs from two sets. Thus, the two Cartesian Products, T×B and B×T, which are formed between the collected B-cell epitopes dataset and the T-cell epitopes dataset can cover substantially all of the possible combination of the known B-cell and T-cell epitopes. The 600 antigens were used to train a neural network that can identify protective antigens. This neural network was used on the Cartesian Product to sieve out 706,970 peptide sequences predicted to be protective antigens. Those 706,970 peptides contain both B-cell epitopes and T-cell epitopes and are protective antigens, referred in this application as the positive vaccine dataset. The same number of peptides randomly bridged by 75 negative T-cell and B-cell epitopes form a negative vaccine dataset.

Network Training

In some embodiments, a multi-layer convolutional neural network (CNN) and a four-layer linear neural network are connected together, forming a deep neural network (DNN) with a two-class output. The positive and negative datasets are annotated by Z-descriptors²⁷, then converted to the same length of 45 vectors with auto cross covariance (ACC) transformation²⁸. Trained by the transformed dataset above, the DNN achieves the classification function to predict whether the input is a viral protective antigens containing both the B-cell epitopes and T-cell epitopes, realizing the ability to directly judge whether a sequence can be a potential peptide vaccine. This DNN is a core part of a rapid vaccine design process of the present framework.

Validation

ROC Curves

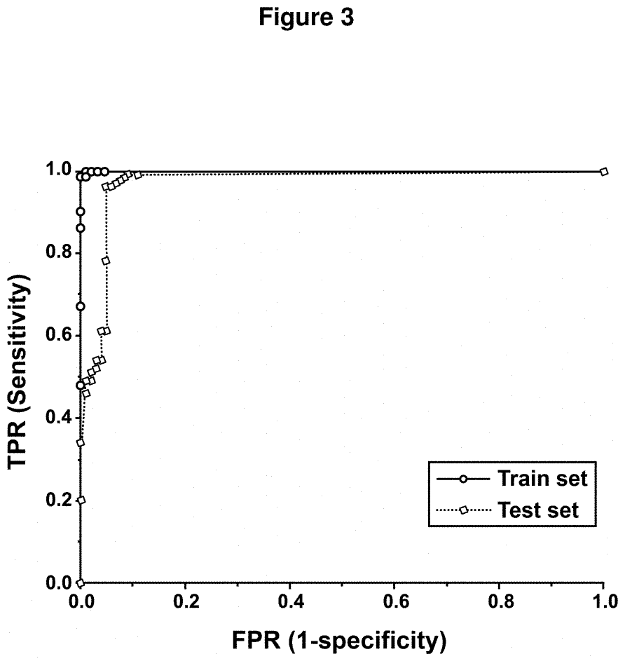

A receiver operating characteristic (ROC) curve is a graphical plot that illustrates the diagnostic ability of a binary classifier system as its discrimination threshold is varied²⁹. ROC curves were used to evaluate the DNN of the present framework. The trained DNN was tested with two datasets, namely a training set and a test set, each of which contains 200 protein sequences. The training set contains 200 proteins randomly selected from the dataset used to train the DNN, with 100 positive and 100 negative protein sequences. Other known B-cell epitopes and T-cell epitopes were also selected and used to form the test set, also with 100 positive and 100 negative protein sequences. The ROC curves are shown in FIG. 3. The area under the ROC curves represent the ability of the present framework to classify potential vaccine subunits and non-potential vaccine subunits. The high area under the ROC curves suggests that the present framework has strong classification ability and high accuracy at most threshold values.

A validation data appears in Table 1. Thresholds are ranged from 0 to 1. The accuracy reported in Table 1 is the greatest value among all thresholds. The sensitivity and specificity values in Table 1 are reported for the case with the highest accuracy. The AUC (Area Under the ROC Curve) value of 0.9703 for the test set indicates the high accuracy of classification of the present systems, methods, and/or vaccines.

TABLE 1
Validation.

| Validation | | | | | |
|---|---|---|---|---|---|
| Specificity % | AUC | Threshold | Accuracy(%) | Sensitivity % | |
| Train set | 0.9999 | 0.32 | 0.995 | 0.99 | 0.99 |
| Test set | 0.9703 | 0.5 | 0.95 | 0.95 | 0.95 |

Vaccine Design Test

The false positive rate (FPR) falls to 0 if the threshold is set to a low value, e.g., 0.0003, since we only care about discarding all the non-candidates. The present framework was used on the 1273 aa spike protein sequence of the CoV. Several (130) vaccine candidates were predicted. BepiPred²², NetMHCpan²³ and Vaxijen³⁰ were used to examine each candidate. All of the candidates contain both T-cell and B-cell epitopes and only 14 of them are predicted by Vaxijen, for example, to be non-protective antigens.

Present Framework

FIG. 2 provides a schematic diagram of the vaccine design process of the present framework. As shown in FIG. 2, a small number of vaccine candidates is predicted, then final vaccine candidates are sieved out by further evaluation, including the T-cell epitope prediction, B-cell epitope prediction, protective antigens prediction, physicochemical analysis. Compared to the popular computational processes, those evaluations are done on a much smaller amount of data, and hence improve efficiency compared to prior processes.

Results

Data Retrieval

The genome sequence of SARS-CoV-2 isolate Wuhan-Hu-1 was retrieved from the NCBI database with accession number MN908947³¹. The protein sequences were retrieved according to their translation. Importantly, the spike protein (protein ID: QHD43416.1) has a length of 1273 amino acids (aa), and the receptor binding domain (RBD) is from 347 to 520aa³². The following descriptions are mainly focused on the spike protein area, though this is not intended to be limiting.

Present Framework Vaccine Candidates Prediction

Overlapping protein fragments with a length of 30aa were generated out of the 1273aa SARs-CoV-2 spike protein sequence. All these 1244 30aa protein sequences were tested with the present framework. Several (130) vaccine candidates are sieved out for further evaluation (See Table 3).

B-Cell Epitope Prediction

B-cell epitopes are portions of antigens binding to immunoglobulin or antibody to trigger the B-cell to provide immune response. Machine-learning approaches train a neural network classifier with a dataset of known B-cell epitopes from experiments to identify whether an input is a B-cell epitope³³. The present framework combines the datasets from online tools including BepiPred²², SVMtrip³⁴, ABCPred³⁵ and LBtope³⁶, to form a B-cell epitope dataset, which covers a more comprehensive range of known B-cell epitopes. These four online tools are all based on supervised learning and are popular among researchers.

In the present framework, a DNN is trained on the B-cell epitope dataset, achieving the ability to predict B-cell epitopes. The overlapping protein sequences of the 130 vaccine candidates were tested with this DNN and 47 sequences were predicted to be potential B-cell epitopes. B-cell epitopes must be located in the solvent-exposed region of the antigens to be possible to combine with the B-cell³³, thus it is essential to predict the surface availability of the structural protein sequence. The surface availability prediction is achieved by Emini tool³⁷. Only the common parts of the two predictions, with 20 B-cell epitopes in all were considered as the results of B-cell epitope prediction (see Table 4). Two of the 20 B-cell epitope candidates were located in the RBD region, which are ‘439-NNLDSKV-445’ and ‘455-LFRKSN-460’. ‘774-QDKNTQ-779’ is the epitope with the highest Emini score of 4.752. The vaccine candidates without those 20 B-cell epitopes were discarded.

T-Cell Epitope Prediction

T-cell provides long-lasting immune response. Machine-learning (ML) methods for T-cell epitope prediction are based on peptide sequences that are known to have the ability to bind with MHC molecules³³. Four popular online ML tools including IL4pred³⁸, NetMHCpan²³, NetMHCIIpan³⁹ and MHC2PRED⁴⁰ were used to predict the T-cell epitopes among the vaccine candidates. The predicted epitopes can cover MHC-1 binders, MHC-2 binders, and Interleukine-4 (IL4) inducing MHC-2 binders³⁸. 26 most common 133 HLA alleles (HLA-A*01:01, HLA-A*02:01, HLA-A*03:01, HLA-A*24:02, HLA-A*26:01, HLA-B*07:02, HLA-B*08:01, 134 HLA-B*27:05, HLA-B*39:01, HLA-B*40:01, HLA-B*58:01, HLA-B*15:01, DRB1-1601, DRB1-1501, DRB1-1401, DRB1-135 1301, DRB1-1201, DRB1-1101, DRB1-1001, DRB1-0901, DRB1-0801, DRB1-0701, DRB1-0401, DRB1-0301, DRB1-0125, DRB1-0101) are tested with the 130 candidates. All the epitopes predicted by the four tools were under consideration. Good peptide vaccines have multiple T-cell epitopes. If the number of the T-cell epitopes in a vaccine candidate is less than 10, it will be discarded, unless it contains multiple B-cell epitopes⁴¹. Some of the vaccine sequences were slightly adjusted to cut down the unnecessary parts without epitopes. For each predicted epitope, an average human-leukocyte-antigen (HLA) score for its MHC-1 and MHC-2 binders was calculated based on the T-cell epitope prediction results as a metric to assess the peptide vaccines. A total of 14 vaccine peptide candidates were selected by the T-cell epitope prediction (see Table 5), among which Vaccine 4, ‘FVFKNIDGYFKIYSKHTPINLVRDLPQGFS’, contains the most T-cell epitopes, has the highest HLA score of 1.644, and are able to bind to 24 HLA alleles. Most of the 14 vaccine candidates show high binding affinity with at least 15 HLA alleles.

Protective Antigen Evaluation

The vaccine peptides must be protective antigens to trigger human protective reactions⁴². The DNN used to sieve viral protective antigens is also used to test those 14 vaccine candidates. All the vaccine pass the test (see Table 6). The antigenicity of the 14 vaccine candidates is also evaluated by the online tools Vaxijen2.0^{30, 43} and the score is shown in Table 6. Vaccine 5 ‘IRGDEVRQIAPGQTGKIADYNYKL’ and Vaccine 7, ‘EILDITPCSFGGVSVITPGTNTSNQVAVLYQ’ are the two peptides with the highest antigenicity. Among the final 14 vaccine candidates, Vaccine 5 ‘IRGDEVRQIAPGQTGKIADYNYKL’ and Vaccine 6 ‘NNLDSKVGGNYNYLYRLFRKSNLKPFE,’ are located in the RBD region.

Toxicity and Physicochemical Properties

A vaccine should have little to no toxicity potential, and physicochemical properties are also important to evaluate how vaccines interact with the environment⁴⁴. A toxicity test may be done by ToxinPred server⁴⁵, for example. ToxinPred is based on a support vector machine (SVM) used to predict toxicity, hydropathicity, hydrophilicity, hydrophobicity, and charge. The results are summarized in Table 7. None of the present candidates were toxic. Hydropathicity, hydrophilicity, and hydrophobicity are three parameters used to evaluate whether a vaccine is hydrophilic in nature. Negative hydrophilicity values indicate that the vaccines can interact with water molecules easily. Hydrophilicity and hydropathicity values show how strong the interactions are and the greater the number is, the stronger the interactions are. The charges of those 14 vaccines are in pH=7 environment. The charge of amino acid will decrease in alkaline environment so usually it is better if the charge values are zero or positive. The ExPASy ProtParam Tool⁴⁶ was used to evaluate the half-life, instability index, pI (Theoretical isoelectric point value) and molecule weight of the 14 vaccines. The results are shown in Table 8 Smaller Instability Index values suggest more stability, most of the vaccines are very stable. pI with alkaline values indicates highly basic existence in nature. Better testing results in this section suggest more practical use of the vaccines.

Allergic Reaction Evaluation

Vaccines should not produce severe allergic reactions. Here AlgPred⁴⁷, which is an SVM based tool, was used to predict the potential allergenicity of the 14 vaccine candidates. The results of allergenicity evaluation are shown in Table 9. The vaccines that are predicted to be potential allergens should be treated carefully for clinical use.

RNA Mutations

As the CoV spreads all over the world, its RNA sequence is going through mutations, translating out different virus proteins. Such mutations can have influences on the epitope based vaccines, since a single amino acid difference can change the epitope prediction results. Interestingly, the 14 present vaccines can tackle the mutations, and the mutations can create new potential vaccine candidates.

The RNA sequence used to translate the spike protein and design the vaccines is from Wuhan, which is also the source of the original virus³¹. The RNA mutations result in three most frequent changes in the spike protein area of the CoV and each of the changes contains one amino acid change48. Table 2 shows the mutation details.

TABLE 2

| Mutations | Occurrence | Regions |
|---|---|---|
| G476S | 3 | Washington |
| V483A | 6 | Washington |
| D614G | 116 | Washington, Los Angeles, New York, South |
| | | America, Europe |

Spike Protein Mutations. Occurrence is the number of isolates that showed the mutation. Region is the origin of the isolates.

The mutation at the 614aa in spike protein from D to G is the most frequent mutations with 116 known isolates⁴⁸. This mutation is very common in many cities in North America. In Europe and South America the D614G mutation occurs in less than 10 isolates. This change has no influence on the 14 vaccine candidates since none of them contain the 614aa. We further investigate the 60aa-length sequence near the 614aa position and no new B-cell epitope is found. The most frequent mutation can be tackled by the 14 vaccines.

At 476aa in spike protein there is a frequent mutation from G to S, which occurs in 3 isolates from Washington DC⁴⁸. This mutation has no influence on the 14 vaccine candidates since none of them contain the 476aa. But this mutation creates an exposed B-cell epitope ‘QASTP’ exactly at the mutated spot. New vaccine candidates can be designed from this mutation, containing two B-cell epitopes. The vaccine sequence is ‘LFRKSNLKPFERDISTEIYQASSTPCNGVE’. Further evaluations of this vaccine are listed in Table 10.

At 483aa in spike protein there is a frequent mutation from V to A, which occurs in 6 isolates from Washington DC⁴⁸. This mutation has no influence on the 14 vaccine candidates since none of them contain the 483aa. But this mutation creates an exposed B-cell epitope ‘GSTPCNGAE’ exactly at the mutated spot. A new vaccine, ‘LFRKSNLKPFERDISTEIYQAGSTPC-NGAE’, can be designed for this mutation, containing two B-cell epitopes, which is very similar to the Vaccine 15. Further Evaluations of this vaccine is listed in Table 11.

Discussion

The present in-silico vaccine design framework (e.g., systems, methods, etc.) has high efficiency and it strongly emphases the multi-epitope in the vaccine peptides. The present framework is an efficient vaccine sieving framework, that utilizes a DNN to rapidly select potential (e.g. the 130) vaccine candidates, introducing a new way to have much higher speed and efficiency in in-silico vaccine design. The present framework can be used to directly predict a potential vaccine peptide sequence by a single neural network without any middle steps. With this framework, the present framework is able to skip at least 95% of unnecessary predictions. Using the present framework to predict the 130 candidates from the virus only takes 1 second, and the number of peptides needed for evaluation is reduced from at least 1300 to 130, so the speed is at least 10 times faster than previous popular in-silico vaccine design approaches, which need to perform an evaluation on all overlapping proteins of a whole virus sequence. In addition, collecting data from online tools and waiting for an online server is very slow. With the present framework, the protein sequences that need to be evaluated can be reduced (e.g., ten times smaller), hence only about 2 hours of time was needed (compared to 20 or more hours with traditional approaches) to finish the vaccine design process.

This approach can be further developed by enhancing the complexity and coverage of the dataset. With the present framework, a part of known epitopes and protective antigens can be selected to form a dataset used to train the DNN. Bridging of one B-cell epitope and one T-cell epitope can be used. With a more comprehensive dataset and more possibilities of epitopes in combination, the present framework is better able to quickly develop a vaccine design. With the present framework, the application of DNN in protein sequences classification shows great potential. Most of the online tools rely on the SVM learning model. In the very popular protective antigens prediction tool Vaxijen³⁰, the AUC of the ROC curve only reached 0.743, which cannot be used to perform accurate predictions. The dataset to train Vaxijen only contains 200 proteins, so it becomes more time consuming and challenge to rely on the SVM model with increasing number of discovered protective antigens. Consequently, the present framework proves that DNN can perform a very accurate prediction with over 700000 different proteins in the dataset.

In an example embodiment, the present framework was used to select the 14 best peptide vaccine candidates from the 130 candidates predicted by the present framework. All of them involve both the B-cell epitopes and T-cell epitopes, providing great potential for the next step COVID-19 vaccine design with actual experiments and clinical studies.

Furthermore, the present framework can be used to trace the RNA mutations of the CoV. Basically the RNA mutations can result in one amino acid change in the spike protein. There are three most frequent mutations, and the 14 vaccines described herein can tackle all of them. Two of those three mutations create two new vaccine candidates because the amino acid change create new epitopes.

Methods

Present Framework Vaccine Candidates Prediction

All the overlapping proteins with a length of 30aa are generated from the 1273aa spike protein sequence of the SARS-CoV-2. All of them were input into the present framework. All the protein sequences that were predicted to be potential peptide vaccines were considered as vaccine candidates.

B-Cell Epitope Prediction

A dataset was collected from the four popular online tools, BepiPred²², SVMtrip³⁴, ABCPred³⁵ and LBtope³⁶, labeled by positive and negative sets. This dataset was annotated by Z-descriptors²⁷ then converted to the same length of 45 vectors with ACC transformation²⁸. A multi-layer convolutional neural network (CNN) and a one-layer linear neural network connect together, forming a deep neural network (DNN) with a two-class output was formed. Trained by the dataset above, the DNN of the present framework has the classification function to predict whether the input is a B-cell epitope under the consideration of all the data from the four online tools. For each vaccine candidate, all the overlapping proteins with length from 4aa to 20aa are tested with this DNN to predict the B-cell epitopes. The entire structural protein sequence is input into the Emini tool³⁷ to predict the protein parts exposed to the surface.

T-Cell Epitope Prediction

T-cell epitope prediction is done to the location in spike protein area that the 130 vaccine candidates cover. By using the four popular online tools including IL4pred³⁸, NetMHCpan²³, NetMHCIIpan³⁹ and MHC2PRED⁴⁰, we can take both MHC-1 and MHC-2 binders²⁵ into consideration and predict the number and locations of the T-cell epitopes. For MHC-1 binders we test all the 9aa overlapping proteins. For MHC-2 binders we test all the 15aa overlapping proteins. We select 26 most common HLA alleles, including HLA-A*01:01, HLA-A*02:01, HLA-A*03:01, HLA-A*24:02, HLA-A*26:01, HLA-B*07:02, HLA-B*08:01, HLA-B*27:05, HLA-B*39:01, HLA-B*40:01, HLA-B*58:01, HLA-B*15:01, DRB1-1601, DRB1-1501, DRB1-1401, DRB1-1301, DRB1-1201, DRB1-1101, DRB1-1001, DRB1-0901, DRB1-0801, DRB1-0701, DRB1-0401, DRB1-0301, DRB1-0125 and DRB1-0101. The final vaccine candidates are selected based on the number of T-cell and B-cell epitopes. For each selected vaccine candidate, an average human-leukocyte-antigen (HLA) score is calculated based on the T-cell epitope prediction results. IL4pred is available at URL:http://crdd.osdd.net/raghava/i14pred/. NetMHCpan is available at URL:http://www.cbs.dtu.dk/services/NetMHCpan/. NetMHCllpan is available at URL:http://www.cbs.dtu.dk/services/NetMHCIIpan/. MHC2PRED is available at URL:http://crdd.osdd.net/raghava/mhc2pred/.

Protective Antigen Evaluation

The DNN used to sieve protective antigens in the present framework is also used for the vaccine candidates sieving. The antigenicity of the final results are evaluated by Vaxijen 2.03°. Vaxijen 2.0 is available at URL:http://www.ddg-pharmfac.net/vaxijen/VaxiJen/VaxiJen.html.

Toxicity and Physicochemical Properties

The toxicity and other important physico-chemical properties of each vaccine candidate is testd by the ToxinPred server⁴⁵ and ExPASy ProtParam Tool⁴⁶. ToxinPred is available at URL:http://crdd.osdd.net/raghava/toxinpred/. ExPASy ProtParam Tool is available at URL:https://web.expasy.org/protparam/.

Side Effects Evaluation

The allergenicity of each vaccine candidate is tested with AlgPred⁴⁷. ExPASy ProtParam Tool is available at URL:https://webs.iiitd.edu.in/raghava/algpred/submission.html.

TABLE 3
Present Framework prediction Results. Here we show the number
of predicted vaccine candidates for each location.

| Location | Start | End | Number of Vaccine Candidates |
|---|---|---|---|
| Location 1 | 6 | 36 | 2 |
| Location 2 | 53 | 104 | 3 |
| Location 3 | 105 | 167 | 8 |
| Location 4 | 206 | 322 | 22 |
| Location 5 | 352 | 585 | 30 |
| Location 6 | 601 | 741 | 19 |
| Location 7 | 751 | 862 | 17 |
| Location 8 | 878 | 981 | 16 |
| Location 9 | 1034 | 1063 | 1 |
| Location 10 | 1057 | 1186 | 12 |
| Location 11 | 1188 | 1218 | 2 |

TABLE 4
B-cell Epitope Prediction Results.

| | | | | | Emini |
|---|---|---|---|---|---|
| Epitope | Start | End | Length | Peptide | Score |
| B-cell 1 | 18 | 32 | 15 | LTTRTQLPPAYTNSF | 1.937 |
| B-cell 2 | 74 | 80 | 7 | NGTKRFD | 2.678 |
| B-cell 3 | 97 | 100 | 4 | KSNI | 1.395 |
| B-cell 4 | 144 | 151 | 8 | YYHKNNKS | 3.544 |
| B-cell 5 | 207 | 211 | 5 | HTPIN | 1.207 |
| B-cell 6 | 406 | 425 | 20 | EVRQIAPGQTGKIADY | 1.775 |
| | | | | NYK | |
| B-cell 7 | 439 | 445 | 7 | NNLDSKV | 1.508 |
| B-cell 8 | 455 | 460 | 6 | LFRKSN | 2.403 |
| B-cell 9 | 601 | 606 | 6 | GTNTSN | 1.888 |
| B-cell 10 | 655 | 660 | 6 | HVNNSY | 1.460 |
| B-cell 11 | 674 | 685 | 12 | YQTQTNSPRRAR | 3.849 |
| B-cell 12 | 774 | 779 | 6 | QDKNTQ | 4.752 |
| B-cell 13 | 786 | 794 | 9 | KQIYKTPPI | 2.243 |
| B-cell 14 | 805 | 816 | 12 | LPDPSKPSKR | 3.136 |
| B-cell 15 | 1035 | 1043 | 9 | GQSKRVDFC | 1.098 |
| B-cell 16 | 1052 | 1058 | 7 | FPQSAPH | 1.001 |
| B-cell 17 | 1109 | 1118 | 10 | FYEPQIITTD | 1.627 |
| B-cell 18 | 1153 | 1170 | 18 | DKYFKNHTSPDVDLGDIS | 1.833 |
| B-cell 19 | 1179 | 1185 | 7 | IQKEIDR | 1.666 |
| B-cell 20 | 1202 | 1206 | 5 | ELGKY | 2.802 |

TABLE 5
B-cell Epitope Prediction Results.

| | | | | MHC-1 | MHC-2 | HLA | HLA | B Epi- |
|---|---|---|---|---|---|---|---|---|
| Vaccine | Start | End | Peptide | Binder | Binder | Allele | Score | tope |
| Vaccine 1 | 18 | 47 | TTRTQLPPAYTNSFTRGVYYPDKVFRSSVL | 9 | 9 | 22 | 1.291 | 1 |
| Vaccine 2 | 71 | 100 | SGTNGTKRFDNPVLPFNDGVYFASTEKSNI | 6 | 10 | 18 | 0.651 | 2 |
| Vaccine 3 | 141 | 170 | LGVYYHKNNKSWMESEFRVYSSANNCTFEY | 9 | 9 | 15 | 0.793 | 1 |
| Vaccine 4 | 192 | 221 | FVFKNIDGYFKIYSKHTPINLVRDLPQGFS | 9 | 14 | 24 | 1.644 | 1 |
| Vaccine 5 | 402 | 425 | IRGDEVRQIAPGQTGKIADYNYKL | 6 | 7 | 11 | 0.591 | 1 |
| Vaccine 6 | 439 | 466 | NNLDSKVGGNYNYLYRLFRKSNLKPFER | 9 | 8 | 13 | 0.948 | 2 |
| Vaccine 7 | 584 | 613 | EILDITPCSFGGVSVITPGTNTSNQVAVLYQ | 5 | 5 | 9 | 0.888 | 1 |
| Vaccine 8 | 655 | 684 | HVNNSYECDIPIGAGICASYQTQTNSPRRA | 3 | 4 | 11 | 0.729 | 2 |
| Vaccine 9 | 773 | 797 | EQDKNTQEVFAQVKQIYKTPPIKDF | 7 | 9 | 19 | 1.294 | 2 |
| Vaccine 10 | 805 | 834 | LPDPSKPSKRSFIEDLLFNKVTLADAGFIK | 8 | 8 | 18 | 0.686 | 1 |
| Vaccine 11 | 1034 | 1063 | LGQSKRVDFCGKGYHLMSFPQSAPHGVVFL | 8 | 4 | 16 | 0.766 | 2 |
| Vaccine 12 | 1094 | 1122 | VFVSNGTHWFVTQRNFYEPQIITTDNTFV | 8 | 8 | 17 | 1.168 | 1 |
| Vaccine 13 | 1156 | 1185 | FKNHTSPDVDLGDISGINASVVNIQKEIDR | 4 | 8 | 15 | 1.064 | 2 |
| Vaccine 14 | 1179 | 1208 | IQKEIDRLNEVAKNLNESLIDLQELGKYEQ | 5 | 6 | 10 | 0.596 | 2 |

TABLE 6
Antigenicity Prediction Results

| | | | | Protective | Vaxijen |
|---|---|---|---|---|---|
| Vaccine | Start | End | Peptide | Antigen | Score |
| Vaccine 1 | 18 | 47 | TTRTQLPPAYTNSFTRGVYYPDKVFRSSVL | Yes | 0.2486 |
| Vaccine 2 | 71 | 100 | SGTNGTKRFDNPVLPFNDGVYFASTEKSNI | Yes | 0.4791 |
| Vaccine 3 | 141 | 170 | LGVYYHKNNKSWMESEFRVYSSANNCTFEY | Yes | 0.3581 |
| Vaccine 4 | 192 | 221 | FVFKNIDGYFKIYSKHTPINLVRDLPQGFS | Yes | 0.4757 |
| Vaccine 5 | 402 | 425 | IRGDEVRQIAPGQTGKIADYNYKL | Yes | 0.9887 |
| Vaccine 6 | 439 | 466 | NNLDSKVGGNYNYLYRLFRKSNLKPFER | Yes | 0.3776 |
| Vaccine 7 | 584 | 613 | EILDITPCSFGGVSVITPGTNTSNQVAVLYQ | Yes | 0.8569 |
| Vaccine 8 | 655 | 684 | HVNNSYECDIPIGAGICASYQTQTNSPRRA | Yes | 0.5673 |
| Vaccine 9 | 773 | 797 | EQDKNTQEVFAQVKQIYKTPPIKDF | Yes | 0.4000 |
| Vaccine 10 | 805 | 834 | LPDPSKPSKRSFIEDLLFNKVTLADAGFIK | Yes | 0.3665 |
| Vaccine 11 | 1034 | 1063 | LGQSKRVDFCGKGYHLMSFPQSAPHGVVFL | Yes | 0.6173 |
| Vaccine 12 | 1094 | 1122 | VFVSNGTHWFVTQRNFYEPQIITTDNTFV | Yes | 0.3387 |
| Vaccine 13 | 1156 | 1185 | FKNHTSPDVDLGDISGINASVVNIQKEIDR | Yes | 0.6035 |
| Vaccine 14 | 1179 | 1208 | IQKEIDRLNEVAKNLNESLIDLQELGKYEQ | Yes | 0.3777 |

TABLE 7
ToxinPred Prediction Results.

| | | SVM | Toxi- | Hydro- | Hydro- | Hydro- | |
|---|---|---|---|---|---|---|---|
| Vaccine | Peptide | Score | city | phobicity | pathicity | philicity | Charge |
| Vaccine 1 | TTRTQLPPAYTNSFTRGVYYPDKVFRSSVL | −0.74 | NT | -0.20 | -0.51 | -0.21 | 3.00 |
| Vaccine 2 | SGTNGTKRFDNPVLPFNDGVYFASTEKSNI | -1.48 | NT | -0.17 | -0.67 | 0.05 | 0.00 |
| Vaccine 3 | LGVYYHKNNKSWMESEFRVYSSANNCTFEY | -1.47 | NT | -0.20 | -0.88 | -0.20 | 0.50 |
| Vaccine 4 | FVFKNIDGYFKIYSKHTPINLVRDLPQGFS | -1.30 | NT | -0.09 | -0.17 | -0.28 | 2.50 |
| Vaccine 5 | IRGDEVRQIAPGQTGKIADYNYKL | -0.43 | NT | -0.24 | -0.78 | 0.29 | 1.00 |
| Vaccine 6 | NNLDSKVGGNYNYLYRLFRKSNLKPFER | -1.69 | NT | -0.34 | -1.16 | 0.18 | 4.00 |
| Vaccine 7 | EILDITPCSFGGVSVITPGTNTSNQVAVLYQ | -1.54 | NT | 0.04 | 0.42 | -0.49 | 2.00 |
| Vaccine 8 | HVNNSYECDIPIGAGICASYQTQTNSPRRA | -0.59 | NT | -0.20 | -0.63 | -0.08 | 0.50 |
| Vaccine 9 | EQDKNTQEVFAQVKQIYKTPPKDF | -1.38 | NT | -0.28 | -1.13 | 0.39 | 0.00 |
| Vaccine 10 | LPDPSKPSKRSFIEDLLFNKVTLADAGFIK | -1.10 | NT | -0.14 | -0.18 | 0.23 | 1.00 |
| Vaccine 11 | LGQSKRVDFCGKGYHLMSFPQSAPHGVVEL | 0.83 | NT | 0.05 | 0.03 | 0.34 | 3.00 |
| Vaccine 12 | VFVSNGTHWFVTQRNFYEPQIITTDNTFV | -1.47 | NT | -0.05 | -0.13 | -0.60 | -0.50 |
| Vaccine 13 | FKNHTSPDVDLGDISGINASVVNIQKEIDR | -0.79 | NT | -0.17 | -0.45 | 0.28 | -1.50 |
| Vaccine 14 | IQKEIDRLNEVAKNLNESLIDLQELGKYEQ | -1.24 | NT | -0.27 | -0.36 | 0.53 | -3.0 |

TABLE 8
ExPASy Protparam Tool Prediction Results.

| | | Half- | | Half- | | Insta- | | | |
|---|---|---|---|---|---|---|---|---|---|
| | | life | | life | | bility | Sta- | | |
| Vaccine | Peptide | (vitro) | | (vivo) | | Index | bility | pI | Weight |
| Vaccine 1 | TTRTQLFPAYTNSFTRGVYYPDKVFRSSVL | 7.2 | h | >20 | h | 34.35 | Yes | 9.99 | 3465.91 |
| Vaccine 2 | SGTNGTKRFDNPVLPFNDGVYFASTEKSNI | 1.0 | h | >20 | h | 45.82 | Yes | 5.84 | 3277.00 |
| Vaccine 3 | LGVYYHKNNKSWMESEFRVYSSANNCTFEY | 5.5 | h | 3 | min | 69.83 | No | 6.75 | 3668.46 |
| Vaccine 4 | FVFKNIDGYFKIYSKHTPINLVRDLPQGFS | 1.1 | h | 3 | min | 18.96 | Yes | 9.40 | 3545.56 |
| Vaccine 5 | IRGDFVRQIAPGQTGKIADYNYKL | 20 | h | 30 | min | 4.41 | Yes | 8.43 | 2706.42 |
| Vaccine 6 | NNLDSKVGGNYNYLYRLFRKSNLKPFER | 1.4 | h | 3 | min | 6.95 | Yes | 9.99 | 3407.27 |
| Vaccine 7 | EILDITPCSFGGVSVITPGTNTSNQVAVLYQ | 1 | h | 30 | min | 8.46 | Yes | 3.67 | 3225.11 |
| Vaccine 8 | HVNNSYECDIPIGAGICASYQTQTNSPRRA | 3.5 | h | 10 | min | 45.3 | Yes | 6.73 | 3267.00 |
| Vaccine 9 | EQDKNTQEVFAQVKQIYKTPPIKDF | 1 | h | 30 | min | 29.50 | Yes | 6.31 | 2995.76 |
| Vaccine 10 | LPDPSKPSKRSFIEDLLFNKVTLADAGFIK | 5.5 | h | 3 | min | 67.50 | No | 8.43 | 3348.34 |
| Vaccine 11 | LGQSKRVDFCGKGYHLMSFPQSAPHGVVFL | 5.5 | h | 3 | min | 38.38 | Yes | 9.20 | 3307.31 |
| Vaccine 12 | VFVSNGTHWFVTQRNFYEPQIITTDNTFV | 100 | h | >20 | h | 17.35 | Yes | 5.32 | 3462.28 |
| Vaccine 13 | FKNHTSPDVDLGDISGINASVVNIQKEIDR | 1.1 | h | 3 | min | 24.99 | Yes | 7.75 | 3283.07 |
| Vaccine 14 | IQKEIDRLNEVAKNLNESLIDLQELGKYEQ | 20 | h | 30 | min | 38.35 | Yes | 4.49 | 3534.46 |

TABLE 9
AlgPred Results

| | | Positive | Negative | | |
|---|---|---|---|---|---|
| | | Predictive | Predictive | SVM | |
| Vaccine | Peptide | Value % | Value % | Score | Allergen |
| Vaccine 1 | TTRTQLPPAYTNSFTRGVYYPDKVFRSSVL | 47.13 | 89.71 | -0.379 | No |
| Vaccine 2 | SGTNGTKRFDNPVLPFNDGVYFASTEKSNI | 87.05 | 71.53 | 0.792 | Potential Allergen |
| Vaccine 3 | LGVYYHKNNKSWMESEFRVYSSANNCTFEY | 70.05 | 80.74 | 0.122 | No |
| Vaccine 4 | FVFKNIDGYFKIVSKHTPINLVRDLPQGFS | 70.05 | 80.74 | 0.128 | No |
| Vaccine 5 | IRGDEVRQIAPGQTGKIADYNYKL | 85.64 | 67.96 | 0.928 | Potential Allergen |
| Vaccine 6 | NNLDSKVGGNYNYLYRLFRKSNLKPFER | 70.05 | 80.74 | 0.008 | No |
| Vaccine 7 | EILDITPCSFGGVSVITPGTNTSNQVAVLYQ | 87.05 | 71.53 | 0.685 | Potential Allergen |
| Vaccine 8 | HVNNSYECDIPIGAGICASYQTQTNSPRRA | 81.83 | 74.03 | 0.578 | Potential Allergen |
| Vaccine 9 | EQDKNTQEVFAQVKQIYKTPPIKDF | 70.05 | 80.74 | 0.058 | No |
| Vaccine 10 | LPDPSKPSKRSFIEDLLFNKVTLADAGFIK | 64.55 | 86.61 | -0.179 | No |
| Vaccine 11 | LGQSKRVDFCGKGYHLMSFPQSAPHGVVFL | 0 | 0 | -0.958 | No |
| Vaccine 12 | VFVSNGTHWFVTQRNFYEPQIITTDNTFV | 54.55 | 86.61 | -0.122 | No |
| Vaccine 13 | FKNHISPDVDLGDISGINASVVNIQKEIDR | 85.64 | 67.96 | 0.805 | Potential Allergen |
| Vaccine 14 | IQKEIDRLNEVAKNLNESLIDLQELGKYEQ | 85.64 | 67.96 | 1.149 | Potential Allergen |

TABLE 10
New Vaccine for Mutation G476S.

| Vaccine 15 | LFRKSNLKPFERDISTEIYQASSTPCNGVE |
|---|---|
| B-cell epitope | LFRKSN, QASTP |
| MHC-1 Binder | 5 |
| MHC-2 Binder | 11 |
| HLA Allele | 21 |
| HLA Score | 0.889 |
| Vaxijen Score | 0.6982 |
| Toxicity | −1.42, NT |
| Half-life | 5.5 h vitro, 3 min vivo |
| Hydrophobicity | −0.42, hydrophilic |
| Instability Index | 22.48, stable |
| pI | 6.25 |
| Mol. Weight | 3430.84 |

TABLE 11
New Vaccine for Mutation V483A

| Vaccine 16 | LFRKSNLKPFERDISTEIYQAGSTPCNGAE |
|---|---|
| B-cell epitope | LFRKSN, GSTPCNGAE |
| MHC-1 Binder | 5 |
| MHC-2 Binder | 10 |
| HLA Allele | 20 |
| HLA Score | 0.754 |
| Vaxijen Score | 0.7624 |
| Toxicity | −1.40, NT |
| Half-life | 5.5 h vitro, 3 min vivo |
| Hydrophobicity | −0.23, hydrophilic |
| Instability Index | 13.23, stable |
| pI | 6.25 |
| Mol. Weight | 3472.76 |

### REFERENCES

1. Wu, J., Leung, K. & Leung, G. Nowcasting and forecasting the potential domestic and international spread of the 2019-nCoV outbreak originating in Wuhan, China: a modelling study. The Lancet 395, 689-697, DOI: https://doi.org/10.1016/S0140-6736(20)30260-9 (2020).
2. Zhou, P., Yang, X., Wang, X. et al. A pneumonia outbreak associated with a new coronavirus of probable bat origin. Nature 579, 270-273, DOI: https://doi.org/10.1038/s41586-020-2012-7 (2020).
3. Dong, E., Du, H. & Gardner, L. An interactive web-based dashboard to track COVID-19 in real time. The Lancet 20, 533-534, DOI: https://doi.org/10.1016/S1473-3099(20)30120-1 (2020).
4. Coronavirus: the first three months as it happened. Nature DOI: https://doi.org/10.1038/d41586-020-00154-w (2020).
5. Shang, W., Yang, Y., Rao, Y. et al. The outbreak of SARS-CoV-2 pneumonia calls for viral vaccines. npj Vaccines 5, DOI: https://doi.org/10.1038/s41541-020-0170-0 (2020).
6. Tay, M. Z., Poh, C. M., Rénia, L. et al. The trinity of COVID-19 immunity, inflammation and intervention. Nat Rev Immunol DOI: https://doi.org/10.1038/s41577-020-0311-8 (2020).
7. Huang, C. et al. Clinical features of patients infected with 2019 novel coronavirus in Wuhan, China. The Lancet 395, 497-506, DOI: https://doi.org/10.1016/50140-6736(20)30183-5 (2020).
8. Chen, Y., Liu, Q. & Guo, D. Emerging coronaviruses: Genome structure, replication, and pathogenesis. J. Med. Virol. 92, 418-423, DOI: https://doi.org/10.1002/jmv.25681 (2020).
9. Gewin, V. On the front lines of the coronavirus-vaccine battle. Nature DOI: https://doi.org/10.1038/d41586-020-01116-y (2020).
10. Callaway, E. The race for coronavirus vaccines: a graphical guide. Nature 580, 576-577, DOI: https://doi.org/10.1038/d41586-020-01221-y (2020).
11. Graham, B. Advances in Antiviral vaccine development. Immunol. Rev. 255, 230-242, DOI: https://doi.org/10.1111/imr.12098 (2013).
12. Gandon, S., Mackinnon, M., Nee, S. & Read, F. Imperfect vaccines and the evolution of pathogen virulence. Nature 414, 751-756, DOI: https://doi.org/10.1038/414751a (2001).
13. Gao, Q. et al. Rapid development of an inactivated vaccine for SARS-CoV-2. bioRxiv DOI: https://doi.org/10.1101/2020.04.17.046375 (2020).
14. Kim, Y. C., Dema, B. & Reyes-Sandoval, A. COVID-19 vaccines: breaking record times to first-in-human trials. Npj Vaccines 5, DOI: https://doi.org/10.1038/s41541-020-0188-3 (2020).
15. Oany, A., Emran, A. & Jyoti, T. Design of an epitope-based peptide vaccine against spike protein of human coronavirus: an in silico approach. Drug Des Devel Ther 1139-1149, DOI: https://doi.org/10.2147/DDDT.S67861 (2014).
16. Feng, Y. et al. Multi-epitope vaccine design using an immunoinformatics approach for 2019 novel coronavirus in China (SARS-CoV-2). bioRxiv DOI: https://doi.org/10.1101/2020.03.03.962332 (2020).
17. Purcell, A., McCluskey, J. & Rossjohn, J. More than one reason to rethink the use of peptides in vaccine design. Nat. Rev. Drug Discov. 6, 404-414, DOI: https://doi.org/10.1038/nrd2224 (2007).
18. Callaway, E. Scores of coronavirus vaccines are in competition—how will scientists choose the best? Nature DOI: https://doi.org/doi:10.1038/d41586-020-01247-2 (2020).
19. Mascola, J. R. & Fauci, A. S. Novel vaccine technologies for the 21st century. Nat Rev Immunol 20, 87-88, DOI: https://doi.org/10.1038/s41577-019-0243-3 (2020).
20. Heinson, A. et al. Enhancing the Biological Relevance of Machine Learning Classifiers for Reverse Vaccinology. Int. J. Mol. Sci. 18, 312, DOI: https://doi.org/10.3390/ijms18020312 (2017).
21. Lan, J., Ge, J., Yu, J. et al. Structure of the SARS-CoV-2 spike receptor-binding domain bound to the ACE2 receptor. Nature DOI: https://doi.org/10.1038/s41586-020-2180-5 (2020).
22. Jespersen, M., Peters, B., Nielsen, M. & Marcatili, P. BepiPred-2.0: improving sequence-based B-cell epitope prediction using conformational epitopes. Nucleic Acids Res. 45, W24-W29, DOI: https://doi.org/10.1093/nar/gkx346 (2017).
23. Nielsen, M. et al. NetMHCpan, a method for quantitative predictions of peptide binding to any HLA-A and -B locus protein of known sequence. PLoS One 2, article e796, DOI: https://doi.org/10.1371/journal.pone.0000796 (2007).
24. Zhu, X. & Goldberg, A. Introduction to Semi-Supervised Learning. Morgan Claypool Publ. DOI: https://doi.org/10.2200/S00196ED1V01Y200906AIM006 (2009).
25. Ahmad, T., Eweida, A. & El-Sayed, L. T-cell epitope mapping for the design of powerful vaccines. Anal Chim Acta 6, 13-22, DOI: https://doi.org/10.1016/j.vacrep.2016.07.002 (2016).
26. Agesen, O. The Cartesian Product Algorithm. 9th Eur. Conf. DOI: https://doi.org/10.1007/3-540-49538-X_2 (1995).
27. Hellberg, S., Sjoestroem, M., Skagerberg, B. & Wold, S. Peptide quantitative structure-activity relationships, a multivariate approach. Am. Chem. Soc. 30, 1126-1135, DOI: https://doi.org/10.1021/jm00390a003 (1987).
28. Wold, S., Jonsson, J., Sjöström, M., Sandberg, M. & Rännar, S. DNA and peptide sequences and chemical processes multivariately modeled by principal component analysis and partial least squares projections to latent structures. Anal Chim Acta 277, 239-253, DOI: https://doi.org/10.1016/0003-2670(93)80437-P (1993).
29. Calders, T. & Jaroszewicz, S. Efficient AUC Optimization for Classification. Knowl. Discov. Databases 4702, DOI: https://doi.org/10.1007/978-3-540-74976-9_8 (2007).
30. Doytchinova, I. A. & Flower, D. R. VaxiJen: a server for prediction of protective antigens, tumour antigens and subunit vaccines. BMC Bioinforma. 8, DOI: https://doi.org/10.1186/1471-2105-8-4 (2007).
31. Wu, F. et al. A new coronavirus associated with human respiratory disease in China. Nature 579, 265-269, DOI: https://doi.org/10.1038/s41586-020-2008-3 (2020).
32. Lu, R. et al. Genomic characterisation and epidemiology of 2019 novel coronavirus: implications for virus origins and receptor binding. The Lancet 395, 565-574, DOI: https://doi.org/10.1016/S0140-6736(20)30251-8 (2020).
33. Sanchez-Trincado, J., Gomez-Perosanz, M. & Reche, P. Fundamentals and Methods for T- and B-Cell Epitope Prediction. J. Immunol. Res. DOI: https://doi.org/10.1155/2017/2680160 (2017).
34. Yao, B., Zhang, L., Liang, S. & Zhang, C. SVMTriP: A Method to Predict Antigenic Epitopes Using Support Vector Machine to Integrate Tri-Peptide Similarity and Propensity. PLoS One 7, e45152, DOI: https://doi.org/10.1371/journal.pone.0045152 (2012).
35. Saha, S. & Raghava, G. P. S. Prediction of continuous B-cell epitopes in an antigen using recurrent neural network. Proteins 65, 40-48, DOI: https://doi.org/10.1002/prot.21078 (2006).
36. Singh, H., Ansari, H. & Raghava, G. P. S. Improved method for linear B-cell epitope prediction using antigen's primary sequence. PLoS One 8, e62216, DOI: https://doi.org/10.1371/journal.pone.0062216 (2013).
37. Almofti, Y., Abd-elrahman, K., Gassmallah, S. & Salih, M. Multi Epitopes Vaccine Prediction against Severe Acute Respiratory Syndrome (SARS) Coronavirus Using Immunoinformatics Approaches. Am. J. Microbiol. Res. 6, 94-114, DOI: https://doi.org/10.12691/ajmr-6-3-5 (2018).
38. Dhanda, S., Gupta, S., Vir, P. & Raghava, G. P. S. Prediction of IL4 inducing peptides. J. Immunol. Res. 2013, 263952, DOI: https://doi.org/10.1155/2013/263952 (2013).
39. Nielsen, M. et al. Quantitative predictions of peptide binding to any HLA-DR molecule of known sequence: NetMHCllpan. PLoS Comput. Biol. 4, article e1000107, DOI: https://doi.org/10.1371/journal.pcbi.1000107 (2008).
40. Bhasin, M. & Raghava, G. P. S. SVM based method for predicting HLA-DRB10401 binding peptides in an antigen sequence. Bioinformatics 20, 421-423, DOI: https://doi.org/10.1093/bioinformatics/btg424 (2004).
41. Patronov, A. & Doytchinova, I. T-cell epitope vaccine design by immunoinformatics. Open Biol. 3, DOI: https://doi.org/10.1098/rsob.120139 (2013).
42. Tahir ul Qamar, M. et al. Epitope-based peptide vaccine design and target site depiction against Middle East Respiratory Syndrome Coronavirus: an immune-informatics study. J Transl Med 17, DOI: https://doi.org/10.1186/s12967-019-2116-8 (2019).
43. Ong, E. et al. Vaxign-ML: supervised machine learning reverse vaccinology model for improved prediction of bacteria protective antigens. Bioinformatics DOI: https://doi.org/10.1093/bioinformatics/btaa119 (2020).
44. Iwasaki, A. & Yang, Y. The potential danger of suboptimal antibody responses in COVID-19. Nat Rev Immunol DOI: https://doi.org/10.1038/s41577-020-0321-6 (2020).
45. Gupta, S. et al. In Silico Approach for Predicting Toxicity of Peptides and Proteins. PLoS ONE 8, e73597, DOI: https://doi.org/10.1371/journal.pone.0073957 (2013).
46. Gasteiger, E. et al. John M. Walker: Protein Identification and Analysis Tools on the ExPASy Server. The Proteomics Protoc. Handb. 571-607, DOI: https://doi.org/10.1385/1592598900 (2005).
47. Saha, S. & Raghava, G. P. S. AlgPred: prediction of allergenic proteins and mapping of IgE epitopes. Nucleic Acids Res. 34, W202-W209, DOI: https://doi.org/10.1093/nar/gk1343 (2006).
48. Banerjee, A. K., Begum, F. & Ray, U. Mutation Hot Spots in Spike Protein of COVID-19. Preprints 2020, 2020040281, DOI: https://doi.org/10.20944/preprints202004.0281.v1 (2020).

In some embodiments, the present system(s) and/or methods may be and/or include a neural network or other model that is trained and configured to predict and/or otherwise determine vaccine candidates. As an example, neural networks may be based on a large collection of neural units (or artificial neurons). Neural networks may loosely mimic the manner in which a biological brain works (e.g., via large clusters of biological neurons connected by axons). Each neural unit of a neural network may be simulated as being connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neural unit itself) may have a threshold function such that the signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free-flowing, with connections interacting in a more chaotic and complex fashion.

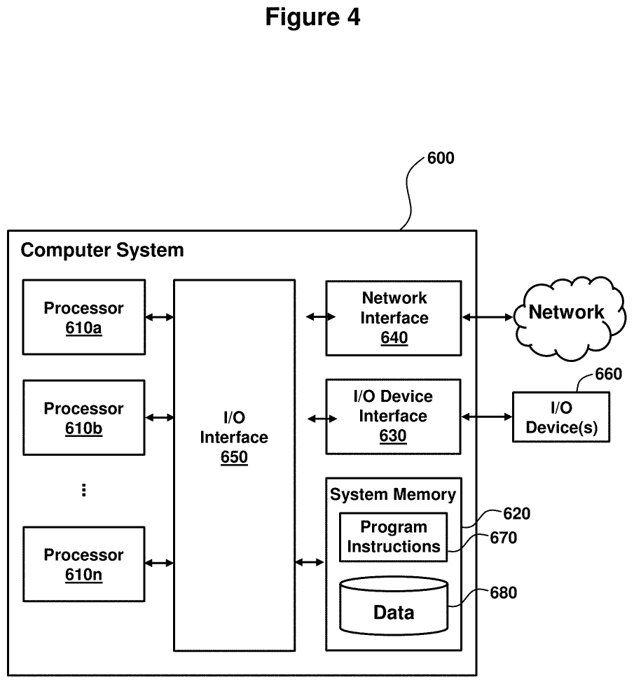

In some embodiments, the present system(s) is executed in a single computing device, or in a plurality of computing devices in a datacenter, e.g., in a service oriented or micro-services architecture. FIG. 4 is a diagram that illustrates an exemplary computing system 600 in accordance with embodiments of the present system. Various portions of systems and methods described herein, may include or be executed on one or more computer systems the same as or similar to computing system 600. For example, the present system itself, a mobile user device, a desktop user device, external resources, and/or other components of the system may be and/or include one more computer systems the same as or similar to computing system 600. Further, processes, modules, processor components, and/or other components of the system described herein may be executed by one or more processing systems similar to and/or the same as that of computing system 600.

Computing system 600 may include one or more processors (e.g., processors 610a-610n) coupled to system memory 620, an input/output I/O device interface 630, and a network interface 640 via an input/output (I/O) interface 650. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing system 600. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 620). Computing system 600 may be a uni-processor system including one processor (e.g., processor 610a), or a multi-processor system including any number of suitable processors (e.g., 610a-610n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computing system 600 may include a plurality of computing devices (e.g., distributed computer systems) to implement various processing functions.

I/O device interface 630 may provide an interface for connection of one or more I/O devices 660 to computer system 600. I/O devices may include devices that receive input (e.g., from a user) or output information (e.g., to a user). I/O devices 660 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 660 may be connected to computer system 600 through a wired or wireless connection. I/O devices 660 may be connected to computer system 600 from a remote location. I/O devices 660 located on remote computer system, for example, may be connected to computer system 600 via a network and network interface 640.

Network interface 640 may include a network adapter that provides for connection of computer system 600 to a network. Network interface may 640 may facilitate data exchange between computer system 600 and other devices connected to the network. Network interface 640 may support wired or wireless communication. The network may include an electronic communication network, such as the Internet, a local area network (LAN), a wide area network (WAN), a cellular communications network, or the like.

System memory 620 may be configured to store program instructions 670 or data 680. Program instructions 670 may be executable by a processor (e.g., one or more of processors 10a-610n) to implement one or more embodiments of the present techniques. Instructions 670 may include modules and/or components of computer program instructions for implementing one or more techniques described herein with regard to various processing modules and/or components. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 620 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. System memory 620 may include a non-transitory computer readable storage medium that may have program instructions stored thereon that are executable by a computer processor (e.g., one or more of processors 610a-610n) to cause the subject matter and the functional operations described herein. A memory (e.g., system memory 620) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times, e.g., a copy may be created by writing program code to a first-in-first-out buffer in a network interface, where some of the instructions are pushed out of the buffer before other portions of the instructions are written to the buffer, with all of the instructions residing in memory on the buffer, just not all at the same time.

I/O interface 650 may be configured to coordinate I/O traffic between processors 610a-610n, system memory 620, network interface 640, I/O devices 660, and/or other peripheral devices. I/O interface 650 may perform protocol, timing, or other data transformations to convert data signals from one component (e.g., system memory 620) into a format suitable for use by another component (e.g., processors 610a-610n). I/O interface 650 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 600 or multiple computer systems 600 configured to host different portions or instances of embodiments. Multiple computer systems 600 may provide for parallel or sequential processing/execution of one or more portions of the techniques described herein.

Those skilled in the art will appreciate that computer system 600 is merely illustrative and is not intended to limit the scope of the techniques described herein. Computer system 600 may include any combination of devices or software that may perform or otherwise provide for the performance of the techniques described herein. For example, computer system 600 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, a television or device connected to a television (e.g., Apple TV™), or a Global Positioning System (GPS), or the like. Computer system 600 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 600 may be transmitted to computer system 600 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present invention may be practiced with other computer system configurations.

Components of the present system may be described as discrete functional blocks, but embodiments are not limited to systems in which the functionality described herein is organized as described. The functionality provided by each of the components may be provided by software or hardware modules that are differently organized than is presently described, for example such software or hardware may be intermingled, conjoined, replicated, broken up, distributed (e.g. within a data center or geographically), or otherwise differently organized. The functionality described herein may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (e.g., content) is said to be supplied or otherwise provided, the information may be provided by sending instructions to retrieve that information from a content delivery network.

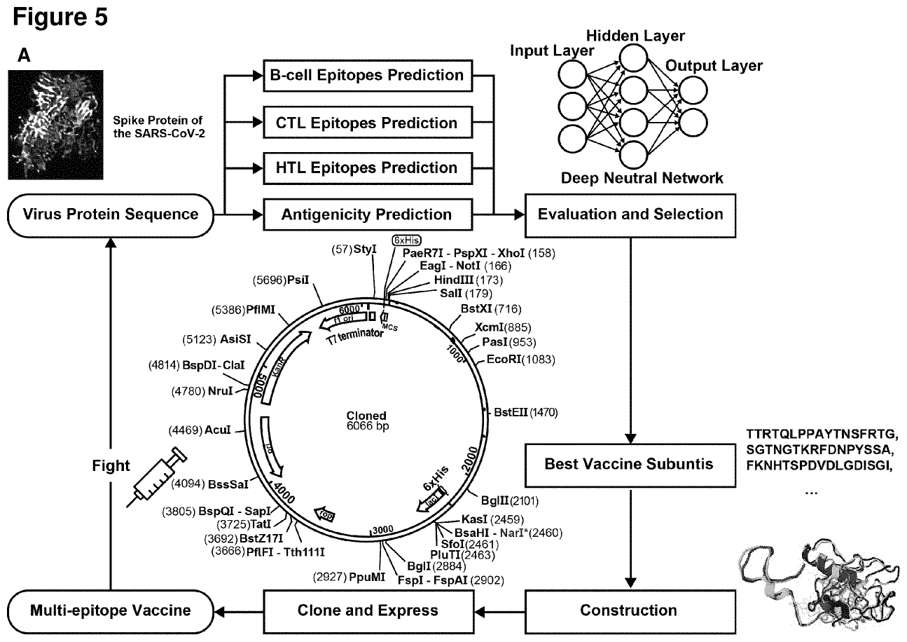

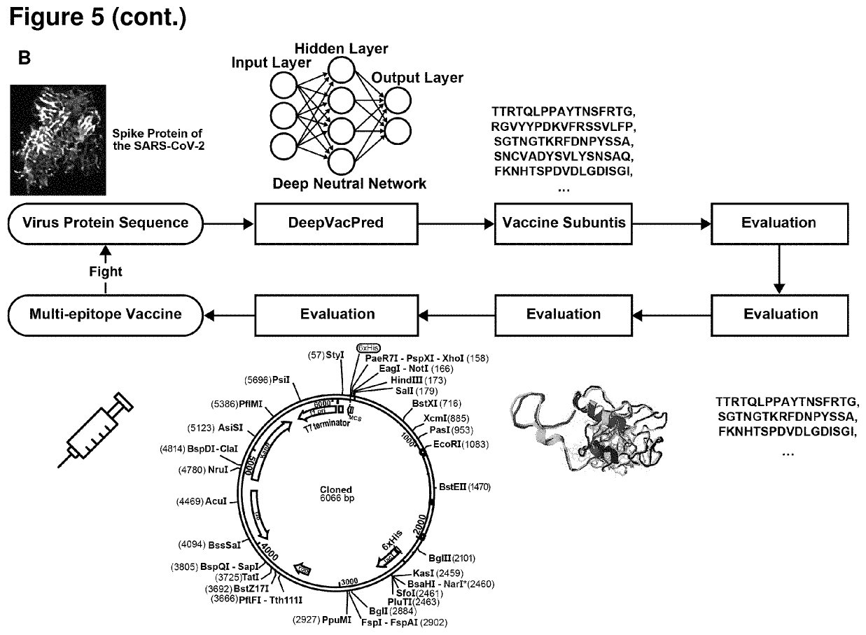

By way of several additional examples, FIG. 5-FIG. 16 illustrate additional details of the systems, methods, and/or vaccines described above. In some cases, one or more of these figures illustrate various data generated with the present systems, methods, and/or vaccines.

Similar to what is shown in FIG. 1 and FIG. 2, FIG. 5 is a schematic diagram of in-silico vaccine design processes. At (A), a traditional in-silico vaccine design process is shown (e.g., similar to what is shown in FIG. 1). With the traditional approach, numerous vaccine design tools are needed. The evaluation and subunits selection is very time consuming No current tool is able to include all the predictions and comprehensively analyze and select out the best vaccine subunits directly. The present framework is illustrated at (B). By replacing the many predictions, evaluations and selections with DNN architecture inside the present framework, we are able to directly predict a very small number of potential vaccine subunits within a second and start the following evaluation and vaccine construction on a much smaller amount of data.

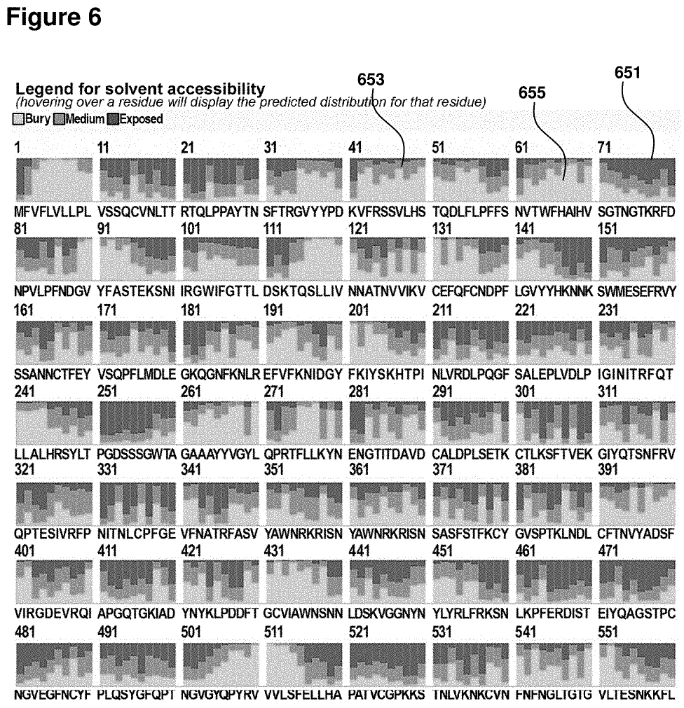

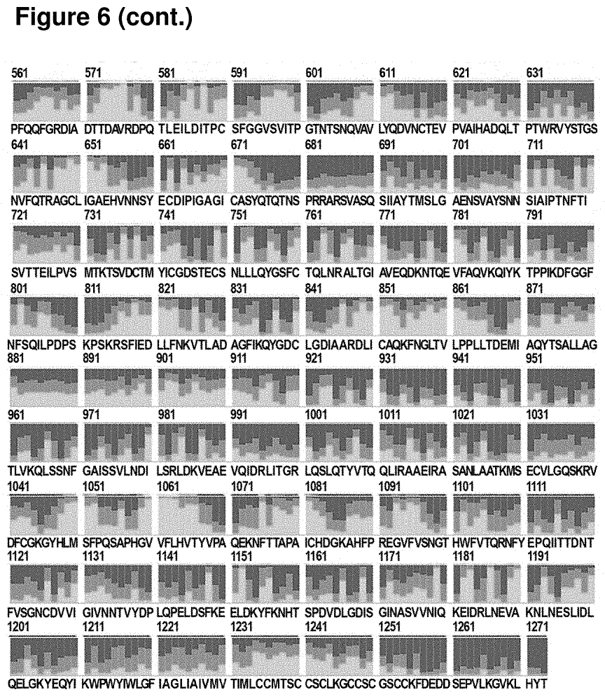

FIG. 6 illustrates the surface accessibility of SARS-CoV-2. The darker areas 651 represent the exposed residues, areas 653 represent the medium exposed residues and areas 655 represent the buried residues. In the SARS-CoV-2 spike protein, the B-cell epitopes in the 14 vaccine subunits are well-exposed according to the surface accessibility prediction, showing good potential that the B-cell receptor is able to interact with the virus to trigger an immune response.

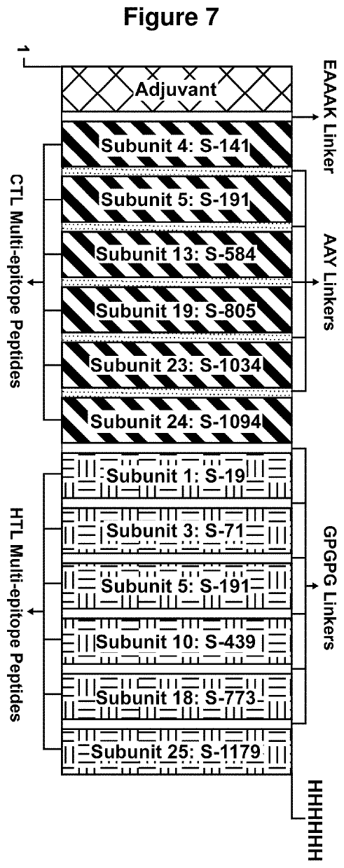

FIG. 7 is a schematic presentation of a present multi-epitope vaccine. The vaccine is constructed by 11 subunits (Subunit 5 is used twice in both CTL and HTL region for its good performance), an adjuvant and a 6×His tag, linked by EAAAK, AAY and GPGPG linkers. The final vaccine consists of 694 amino acid residues. It contains 16 B-cell epitopes, 82 CTL epitopes and 89 HTL epitopes.

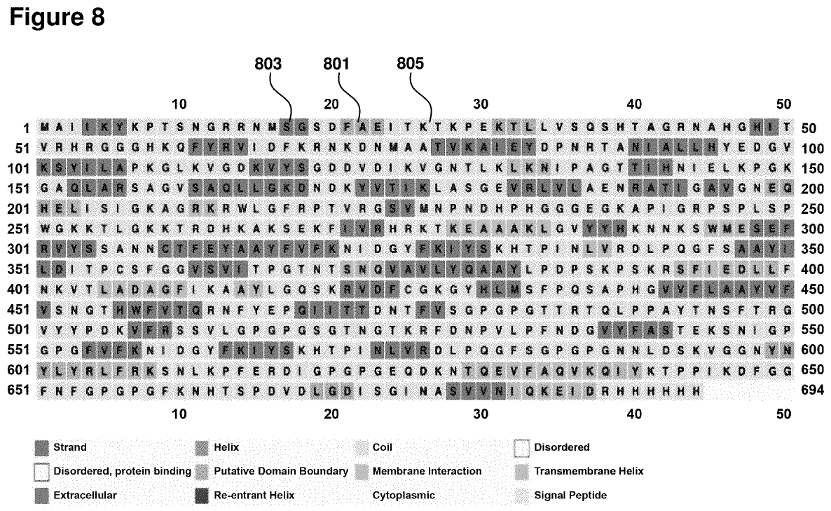

FIG. 8 is a graphical representation of secondary structure features. The alpha helix residues are shown by 801, the beta strand residues are shown by 803 and the coil residues are shown by 805. The predicted secondary structure indicates that the final vaccine constitutes 10.8% alpha helix, 24.6% beta strand, and 64.6% coil, for example.

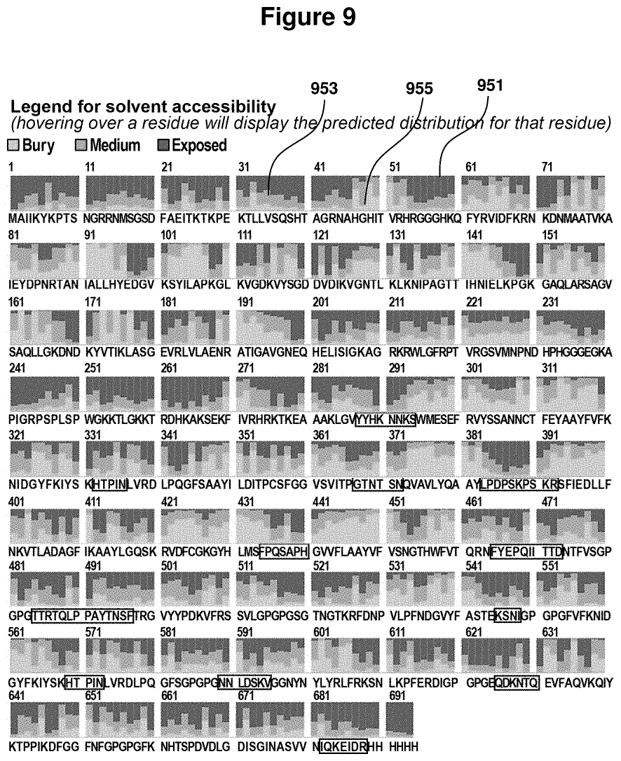

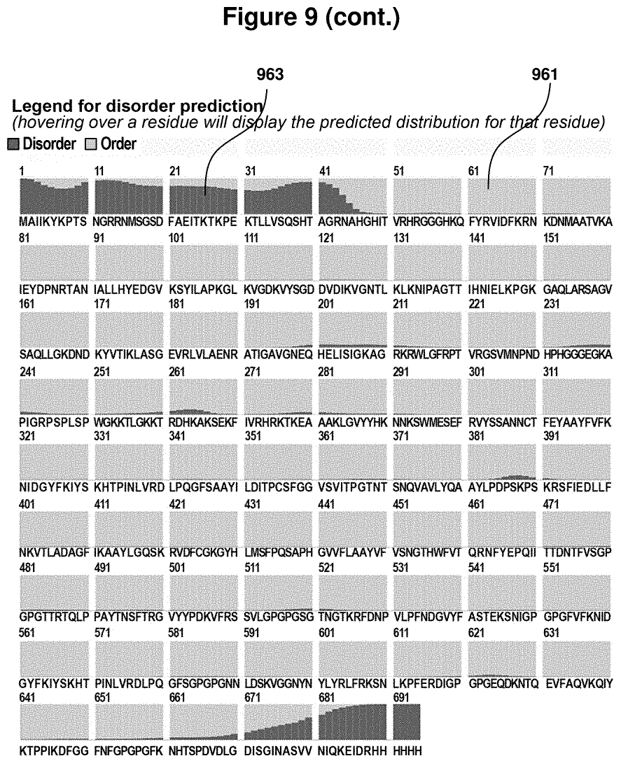

FIG. 9 illustrates solvent accessibility and disorder regions prediction results. In the solvent accessibility prediction results, 951 represents the exposed residues, 953 represents the medium exposed residues and 955 represents the buried residues. The peptides marked in boxes are B-cell epitopes. The prediction results show that the B-cell epitopes in the final vaccine have good surface accessibility and they are not close to each other. In the disorder regions prediction results, the ordered regions are in one color 961 while the disordered regions are in a second color 963. A total of 60 residues (8%) are in disordered regions, showing good order in structure.

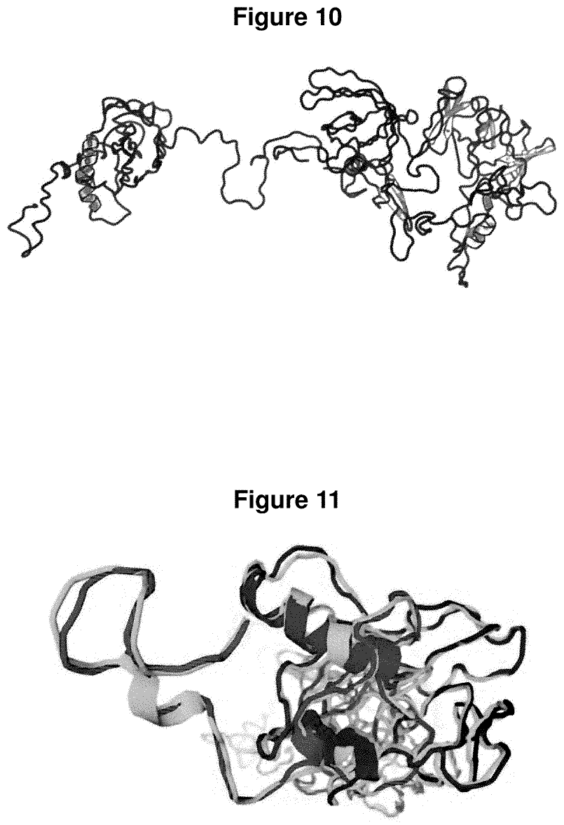

FIG. 10 illustrates present vaccine 3D structure modeling (e.g., by RaptorX) based on the template with PDB ID 3j3vC. All the 694 amino acids in the present vaccine are modeled. The P-value of this model is 4.13×10-14 and this very low value indicates high quality of this 3D model. The unnormalized Global Distance Test (uGDT) score of this model is 506 (>50), indicating good absolute model quality.

FIG. 11 illustrates a refined vaccine 3D structure model (e.g., by GalaxyRefine) for the present vaccine. This model has a Global Distance Test-High Accuracy (GDT-HA) score of 0.900, a Root Mean Square Deviation (RMSD) score of 0.580, a MolProbity score of 2.618, a clash score of 33.5 and a Ramachandran plot score of 87.5%, showing very good overall model quality.

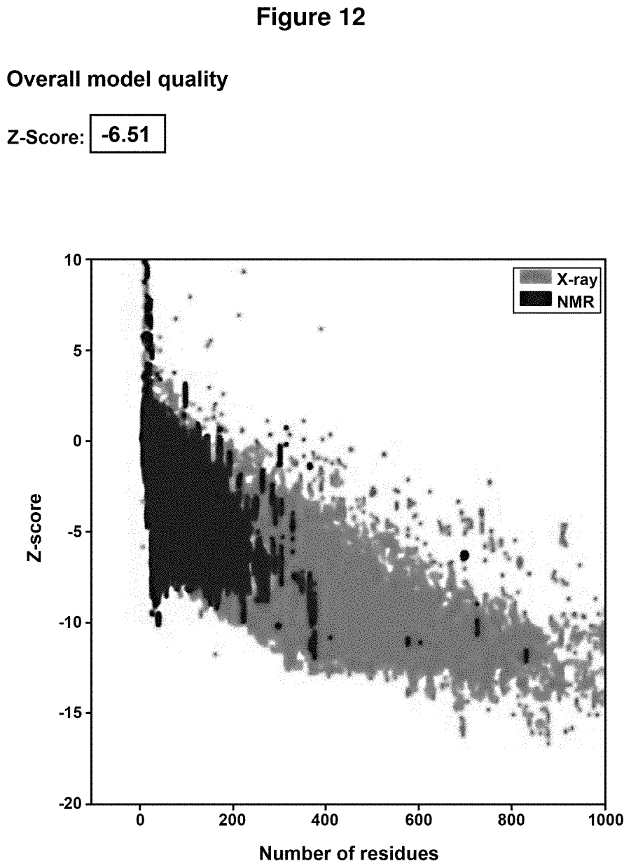

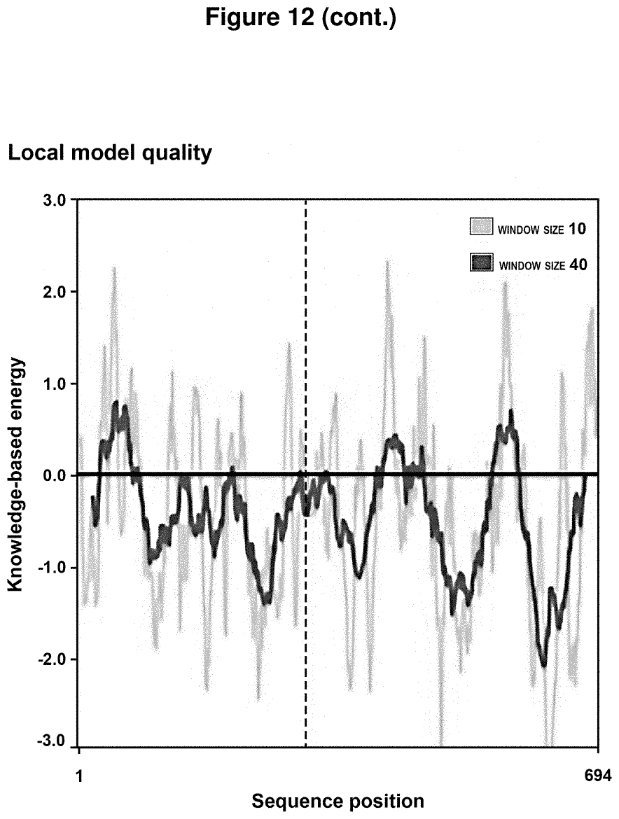

FIG. 12 illustrates vaccine 3D structure validation (e.g., by ProSA-web) for the present vaccine. The Z-score of the refined model is −6.51 which is lying inside the score range. ProSA-web also plots the residues scores to check the local model quality and the negative values suggest no erroneous parts of the model structure.

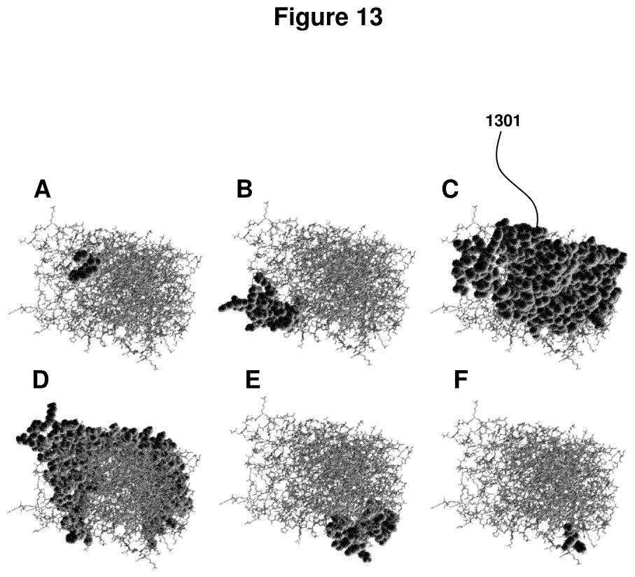

FIG. 13 illustrates 3D models of six predicted conformational B-cell epitopes in the present (refined) vaccine structure. Parts 1301 (e.g., spherical shapes in a-f) are the conformational B-cell epitopes and the other (mesh looking) parts are the rest of the residues. Image (a) represents three residues with a score of 0.963. Image (b) represents 30 residues with a score of 0.757. Image (c) represents 167 residues with a score of 0.711. Image (d) represents 161 residues with a score of 0.688. Image (e) represents 23 residues with a score of 0.59. Image (f) represents three residues with a score of 0.531.

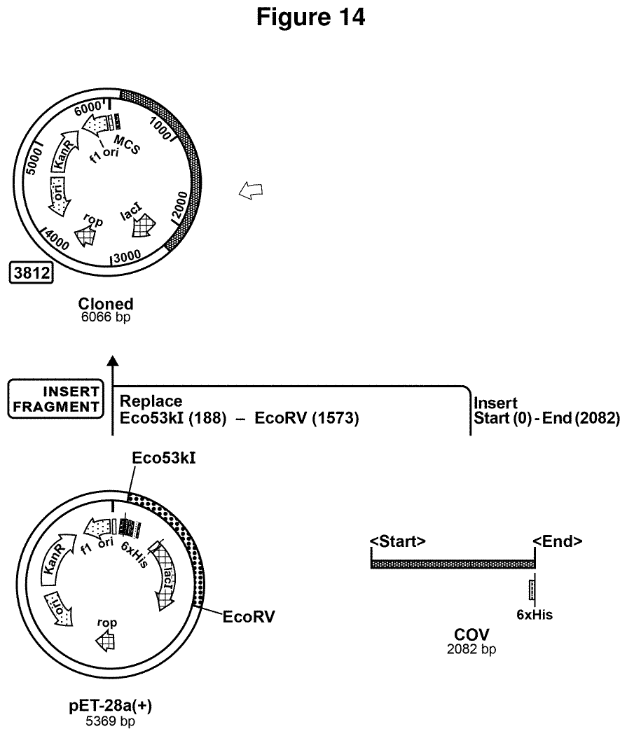

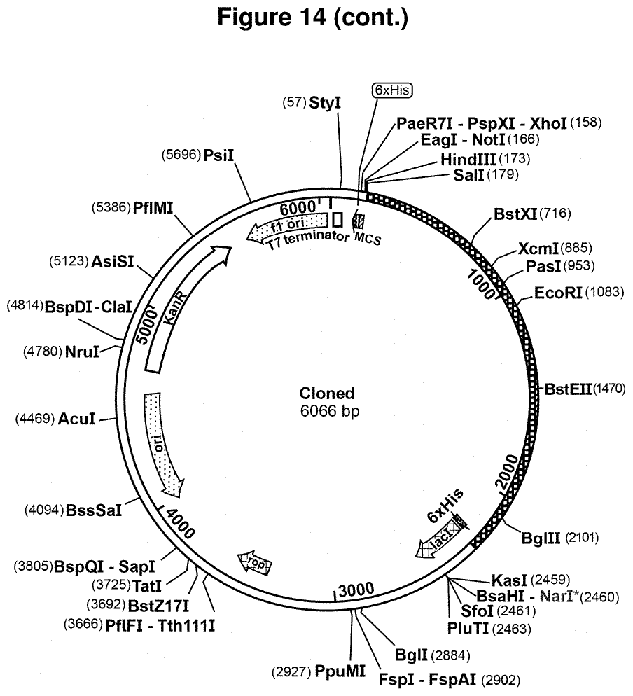

FIG. 14 illustrates vaccine in-silico cloning into the pET28a(+) vector. The codon sequence of the final vaccine is a 2082 bp gene sequence (e.g., generated by the JCat server). The pET28a(+) expression vector is also shown. The codon sequence is inserted between Eco53KI (188) and EcoRV (1573), forming a clone with a total length of 6066 bp. This image, for example, was created by SnapGene 5.1.5 software (from Insightful Science; available at https://www.snapgene.com).

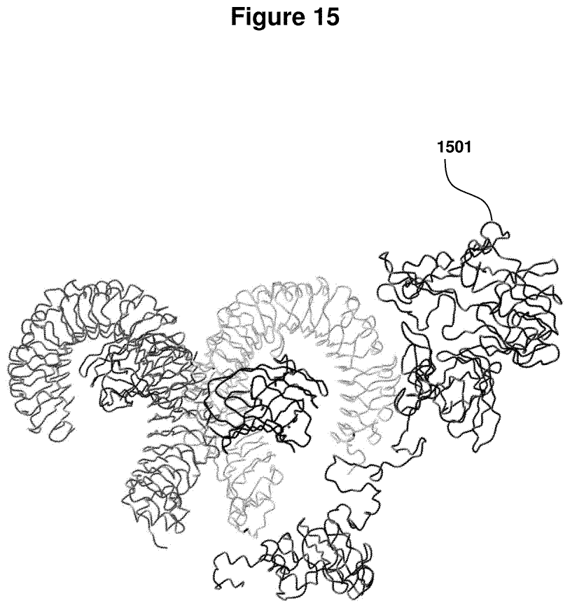

FIG. 15 illustrates the docked complex of the vaccine model and the TLR4 immune receptor. The vaccine protein is represented by 1501, and the rest of the residues are the TLR4 receptor. The lowest energy score of this complex model is −1311.5, indicating good binding affinity.

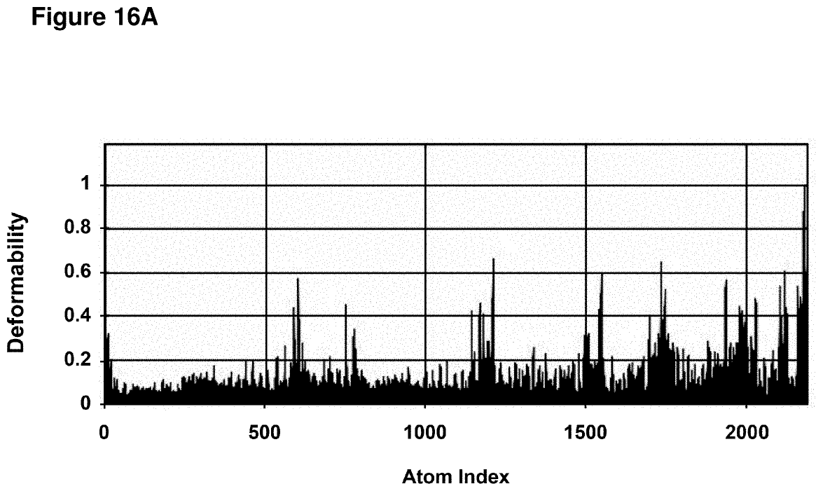

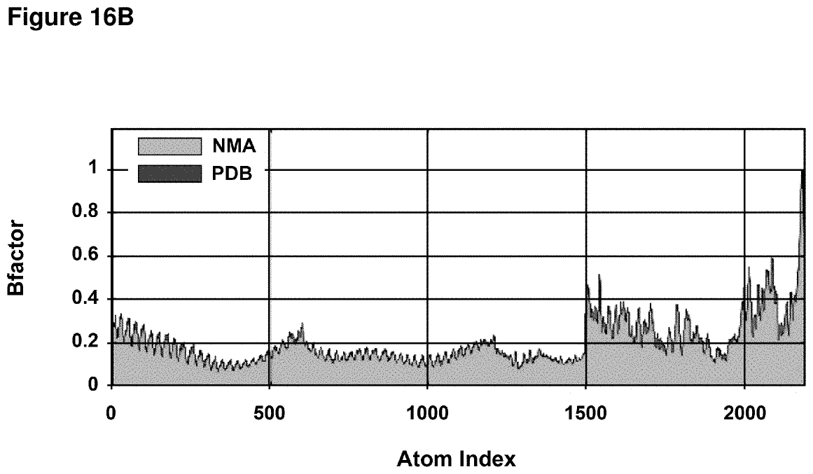

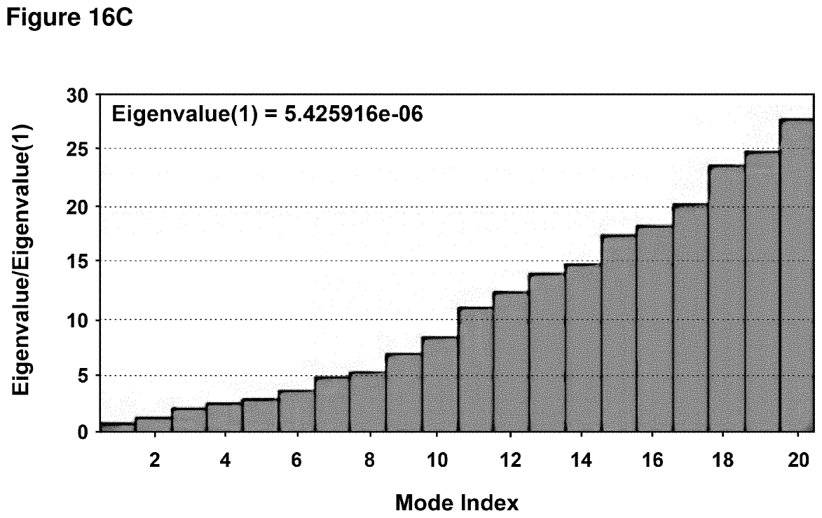

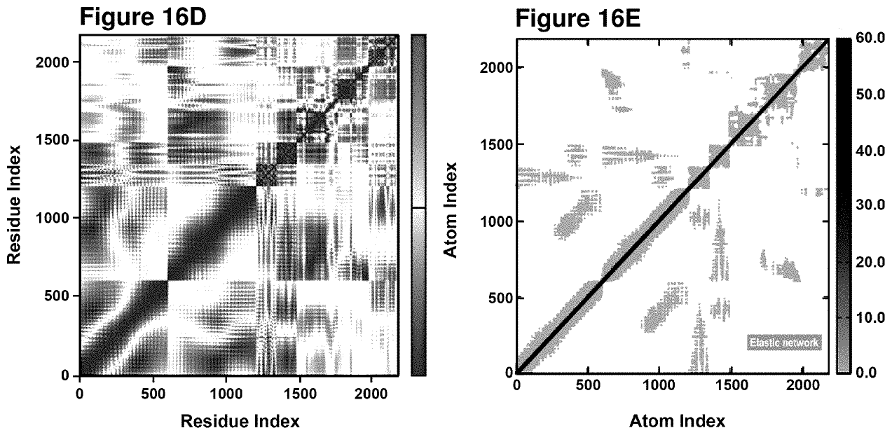

FIG. 16 illustrates a molecular dynamics simulation of the vaccine-TLR4 docked complex. Image (a) shows a main-chain deformability simulation, the hinges are regions with high deformability. Image (b) shows B-factor values calculated by normal mode analysis, quantifying the uncertainty of each atom Image (c) shows the eigenvalue of the docked complex, showing the energy required to deform the structure. Image (d) shows the covariance matrix between pairs of residues. Image (e) shows the elastic network model, suggesting the connection between atoms and springs. The springs are more rigid if the image is darker.

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

The phrase “induced by”, encompasses “worsened by”, “aggravated by”, “exacerbated by”, and/or “magnified by”, unless clearly indicated otherwise.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in some embodiments or aspects of the methods disclosed herein, some materials and/or method steps are excluded. Thus, even though the invention is generally not expressed herein in terms of what the invention does not include aspects that are not expressly excluded in the invention are nevertheless disclosed herein.

Some embodiments of the technology described herein suitably can be practiced in the absence of an element not specifically disclosed herein. Accordingly, in some embodiments the term “comprising” or “comprises” can be replaced with “consisting essentially of” or “consisting of” or grammatical variations thereof. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

1. A composition comprising one or more peptides comprising 5 or more contiguous amino acids of a peptide selected from Tables 3-11.

2. A composition comprising one or more peptides having at least 95% identity to a peptide selected from Tables 3-11.

3. The composition of claim 1, wherein the one or more peptides comprises four or more peptides.

4. The composition of claim 1, comprising a peptide selected from LTTRTQLPPAYTNSF; NGTKRFD; KSNI; YYHKNNKS; HTPIN; EVRQIAPGQTGKIADYNYK; NNLDSKV; LFRKSN; GTNTSN; HVNNSY; YQTQTNSPRRAR; QDKNTQ; KQIYKTPPI; LPDPSKPSKR; GQSKRVDFC; FPQSAPH; FYEPQIITTD; DKYFKNHTSPDVDLGDIS; IQKEIDR; and ELGKY.

5. The composition of claim 1, comprising a peptide selected from Table 4.

6. The composition of claim 1, comprising a peptide selected from Table 5.

7. The composition of claim 1, comprising a peptide selected from Table 6.

8. The composition of claim 1, comprising a peptide selected from Table 7.

9. The composition of claim 1, comprising a peptide selected from Table 8.

10. The composition of claim 1, comprising a peptide selected from Table 9.

11. The composition of claim 1, comprising a peptide selected from Table 10.

12. The composition of claim 1, comprising a peptide selected from Table 11.

13. The composition of claim 1, comprising an adjuvant.

14. The composition of claim 1, wherein the composition induces an immune response in a subject.

15. The composition of claim 14, wherein the immune response comprises generation of one or more antibodies that specifically bind to SARS-CoV-2.

16. The composition of claim 1, wherein the composition is a pharmaceutical composition.

17. The composition of claim 1, wherein the composition is a vaccine.

18. A method of inducing an immune response in a subject comprising administering the composition of claim 1 to a subject.

19. The method of claim 18, wherein the method comprises reducing, inhibiting, mitigating or preventing infections from SARS-CoV-2.

20. The method of claim 18, wherein the method comprises reducing, inhibiting, mitigating or preventing one or more symptoms, or the severity of one or more symptoms of an infection from SARS-CoV-2.

21-38. (canceled)
